(19) United States
(12) Patent Application Publication
(10) Pub. No.: US 2021/0113560 A1
(43) Pub. Date: Apr. 22, 2021

(54) METHOD OF TREATING FIBROTIC DISEASE

(71) Applicant: BLADE THERAPEUTICS, INC., South San Francisco, CA (US)

(72) Inventors: Brad Owen Buckman, Oakland, CA (US); Prabha Ibrahim, Mountain View, CA (US); P T Ravi Rajagopalan, San Ramon, CA (US)

(21) Appl. No.: 17/041,611

(22) PCT Filed: Mar. 25, 2019
(86) PCT No.: PCT/US2019/023917

§ 371 Date: Sep. 25, 2020

Related U.S. Application Data
(60) Provisional application No. 62/649,428, filed on Mar. 28, 2018.

(51) Int. Cl. A61K 31/506; A61K 31/4725; A61K 31/433; A61K 31/4155; A61K 31/4439; A61K 31/166; A61K 31/4015; A61K 31/421; A61K 31/415; A61K 31/27; A61K 31/42; A61K 31/4045; A61K 31/497; A61K 31/423

(52) U.S. Cl. CPC A61K 31/506; A61K 31/4725; A61K 31/433; A61K 31/4155; A61K 31/4439; A61K 31/166; A61K 31/423; A61K 31/421; A61K 31/415; A61K 31/27; A61K 31/42; A61K 31/4045; A61K 31/497; A61K 31/4015

(57) ABSTRACT

Disclosed herein are methods of treating fibrotic disorders by administering compounds selective for CAPN1, CAPN2, and/or CAPN9 such that side effects, off pathway interactions, and/or toxicities are minimized Such methods may, for example, minimize unintended effects of therapeutic compounds by providing dosing and dosage forms that minimize the level of unbound drug within the relevant tissues of a patient undergoing treatment.

### BACKGROUND

### Field

The present disclosure relates to the field of drug delivery. The disclosure contemplates methods of administering specific compounds of pharmaceutical interest, such as calpain inhibitors, to patients in a manner that will minimize toxicity, reduce side effects, and improve patient compliance.

### Background

Fibrotic disease accounts for an estimated 45% of deaths in the developed world but the development of therapies for such diseases is still in its infancy. The current treatments for fibrotic diseases, such as for idiopathic lung fibrosis, renal fibrosis, systemic sclerosis, and liver cirrhosis, are few in number and only alleviate some of the symptoms of fibrosis while failing to treat the underlying cause.

Despite the current limited understanding of the diverse etiologies responsible for these conditions, similarities in the phenotype of the affected organs, across fibrotic diseases, strongly support the existence of common pathogenic pathways. At present, it is recognized that a primary driver of fibrotic disease is a high transforming growth factor-beta (TGFβ) signaling pathway which can promote the transformation of normally functioning cells into fibrosis-promoting cells, which secrete large amounts of extracellular matrix proteins and matrix degrading enzymes, resulting in the formation of scar tissue and eventual organ failure. This cellular process is transformative and termed “myofibroblast differentiation” (which includes Epithelial-to-Mesenchymal Transition (EpMT) and its variations like Endothelial-to-Mesenchymal Transition (EnMT) and Fibroblast-to-Myofibroblast Transition (FMT)). This process is a major target for the treatment of fibrotic diseases. Myofibroblast differentiation has also been shown to occur within cancer cells that have been chronically exposed to high TGFβ, causing stationary epithelial cells to become motile, invasive, and metastasize. Thus, within the context of cancer, the signaling has been documented to associate with the acquisition of drug resistance, immune system evasion, and development of stem cell properties.

Unfortunately, TGFβ is a pleiotropic cytokine with many physiological functions such that global suppression of TGFβ signaling was also associated with severe side effects. Additionally, current data suggests that such proximal inhibition may be vulnerable to pathologic workaround strategies (i.e., due to redundancy or compensation), that would limit the utility of such drugs. Further complicating matters is that, in cancer, TGFβ signaling early on functions as an anti-tumorigenic growth inhibitor but later becomes tumor promoting and is another reason why selective inhibition of pathogenic elements of signaling is so strongly desired. In light of these inherent limitations, current treatment strategies have refocused on identification and inhibition of critical distal events in TGFβ signaling, which in theory would preferentially target the pathologic, but not physiological functions of TGFβ signaling.

It is thus necessary to target pathologic aspects of TGFβ signaling in a highly precise manner in order to achieve therapeutic efficacy in fibrotic disease without engendering dangerous side effects or toxicities. It is well known that the efficacy of a pharmaceutical compound often depends on the affinity and specificity with which it binds to its target, such as a receptor, a signaling molecule, or an enzyme. Low affinity binding can lead to dissociation from the target, and thus to too-rapid removal of the unbound compound from its site of action. High affinity binding is, therefore, often preferred. However, even in a scenario of high-affinity binding, dissociation of the compound from its target, or the presence of excess amounts of the compound, as necessary to provide optimal receptor binding at equilibrium, provides significant amounts of any given compound in the surrounding medium, providing free (unbound) drug which may then participate in off-pathway effects, such as occur when a compound binds to a receptor or site other than its intended target, or in toxicity, in which free (unbound) drug interacts harmfully with receptors in non-target tissues, or is modified by a patient's own enzymes into toxic byproducts. The presence of side effects or toxicities can complicate therapy, of course, as well as reducing patients' willingness to continue taking a drug, such that patient compliance with dosing prescriptions is jeopardized. It would be desirable, therefore, to provide mechanisms for dosing or administering therapeutic compounds such that binding to their target is increased, while at the same time, the amount of the free, unbound compound, is minimized.

The binding affinity of a compound for its target receptor is often expressed as its equilibrium dissociation constant, K_{D}. As conventionally understood, compounds with a low K_{D} represent compounds with higher levels of binding to their target receptors, and thus are considered to be better candidates for therapeutic applications. In the simplest case, K_{D} represents the combination of the rate off association of a free compound with its target (often referred to as the “On Rate”, k_{[On]}) and the rate of dissociation of a bound compound from its target (often referred to as the “Off Rate”, k_{[Off]}), according to the relationship, K_{D}=k_{[Off]}/k_{[On]}. Thus, an increase in binding affinity may reflect an enhancement in the rate at which a compound binds its target (increased k_{[On]}), or a reduction in the rate at which a compound becomes unbound (increased k_{[Off]}). Compounds with a higher “on rate” will appear to have an enhanced equilibrium binding affinity, but due to the presence of unbound compound, due to dissociation as well as administration of said compound at levels sufficient to maintain the equilibrium, will often maintain sufficient amounts of unbound compound to lead to toxic effects or side effects due to off-pathway binding or off-target interactions. On the other hand, a compound with a decreased “off rate” can be administered at lower levels while maintaining its effect over time, due to the increased duration of the binding to the target. In this case, it can be possible to administer the compound at lower levels, to administer the compound less frequently, or to administer the compound such that unbound compound “washes out” of the surrounding medium, leaving only the therapeutically relevant (target-bound) molecules in the body to act on the patient.

Accordingly, there is a need for methods of administering therapeutic compounds that have low off-rates with respect to their therapeutic targets, such that dosing levels can be reduced, with concomitant reductions in side effects and toxicity, as well as possible beneficial effects on patient compliance and outcomes. Within the context of the fibrotic diseases, a need exists for methods of administering drugs targeting TGFβ signaling in highly specific ways, thus limiting neoplasia, cancer promotion, immune effects, or any of the other side effects of global modulation of TGFβ signaling.

### SUMMARY

The present disclosure provides a method of treating a disease or condition, comprising first, administering for a first number of days to a subject in need thereof a first daily amount of one or more compounds having the structure of the formula:

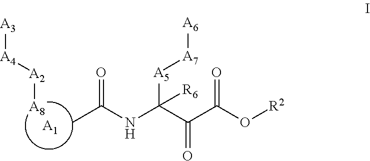

or a pharmaceutically acceptable salt thereof, wherein:

A₁ is selected from the group consisting of optionally substituted 5-10 membered heterocyclyl; optionally substituted 5-, 8-, or 9-membered heteroaryl; and optionally substituted C₃₋₁₀ carbocyclyl;

A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —C≡CH—, —OC(O)NH—, —NHC(O)NH—, —NHC(O)O—, —NHC(O)—, —NHC(S)NH—, —NHC(S)O—, —NHC(S)—, and single bond;

A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;

when A₂ and A₄ are single bond, A₃ is directly attached to A₈;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —OC₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted. C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁸ is attached;

A₈ is a ring member of A₁ and is s the group consisting of C and N;

R is independently selected from —H, optionally substituted C₁₋₄ alkyl, optionally substituted C₁₋₈ alkoxyalkyl, optionally substituted 2- to 5-membered polyethylene optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R² is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R⁶ is independently selected from —H and optionally substituted C₁₋₄ alkyl; and

each n is independently selected to be an integer from 0 to 3.

or any combination thereof; or pharmaceutically acceptable salts thereof and

then, ceasing administration of the compound or administering a second daily amount of the compound for a second number of days, wherein the second daily amount of the compound is less than, greater than, or the same as the first daily amount; and then administering a third daily amount of the compound for a third number of days to the subject.

The present disclosure further provides a method of treating a disease or condition, comprising the steps of, first, administering for a first number of days to a subject in need thereof a first daily amount of a one or more compounds having the structure of the formula:

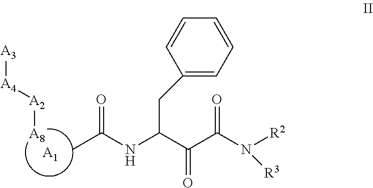

or a pharmaceutically acceptable salt thereof, wherein:

A₁ is selected from the group consisting of optionally substituted 5-10 membered heterocyclyl provided the 5-10-membered heterocyclyl is not substituted with oxo; optionally substituted 5-, 8-, or 9-membered heteroaryl; and optionally substituted C₃₋₁₀ carbocyclyl;

A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —C(═S)—, —C(═O)—, —NR—, —CH═CH—, —C≡C, —OC(O)NH—, —NHC(O)NH—, —NHC(O)O—, —NHC(O), —NHC(S)NH—, —NHC(S)O—, —NHC(S)—, and single bond;

A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;

when A₂ and A₄ are single bond, A₃ is directly attached to A₈;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted. C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;

A₈ is a ring member of A₁ and is selected from the group consisting of C and N;

R is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R² and R³ are independently selected from optionally substituted C₁₋₄ alkyl, optionally substituted. C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and

R⁶ is independently selected from —H and optionally substituted C₁₋₄ alkyl; and each n is independently selected to be an integer from 0 to 3;

or any combination thereof; or pharmaceutically acceptable salts thereof; and then, ceasing administration of the compound or administering a second daily amount of the compound for a second number of days, wherein the second daily amount of the compound is less than, greater than, or the same as the first daily amount; and then, administering a third daily amount of the compound for a third number of days to the subject.

The present disclosure further provides a method of treating a disease or condition, comprising the steps of, first, administering for a first number of days to a subject in need thereof a first daily amount of a compound having a structure selected from the group consisting of:

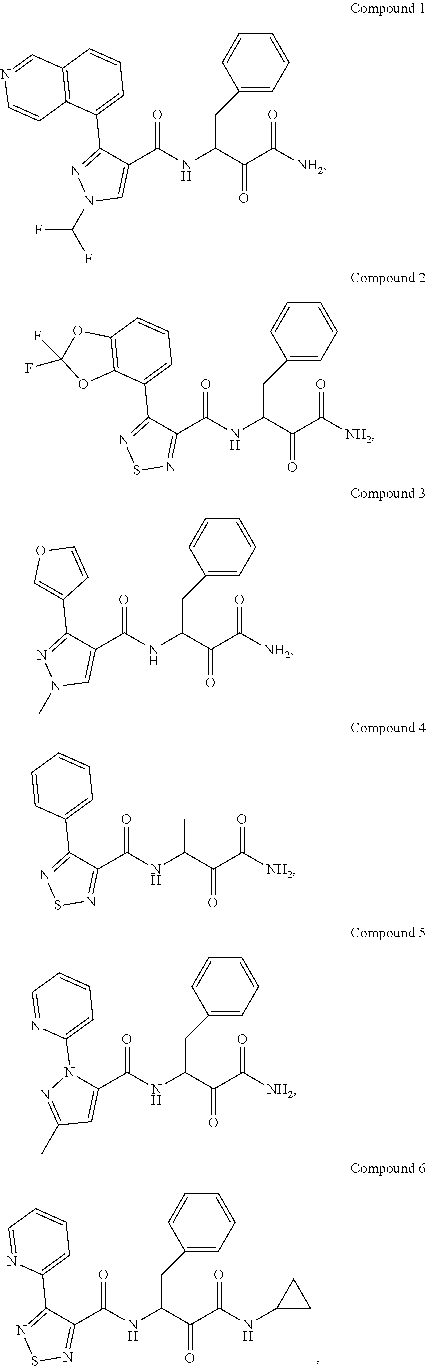

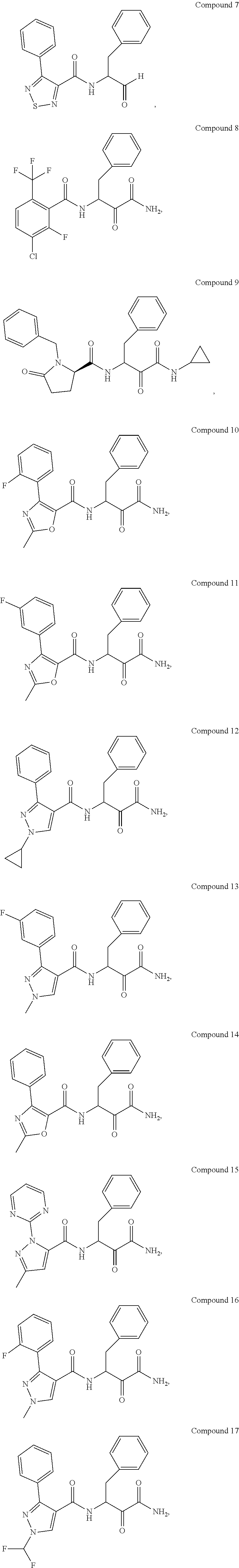

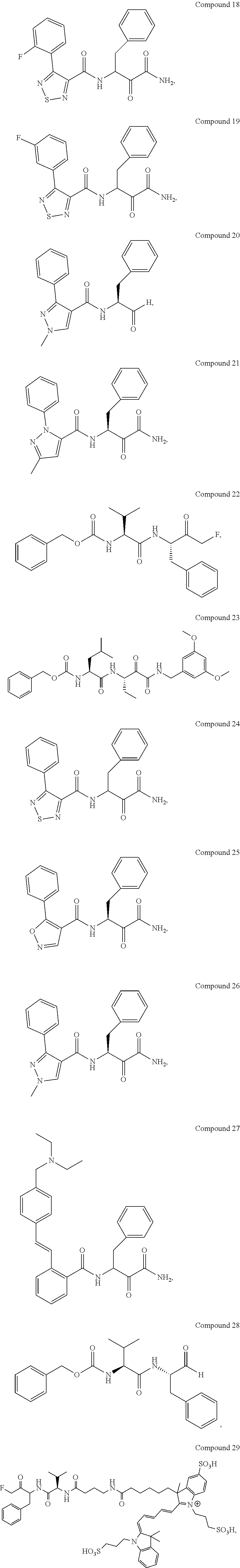

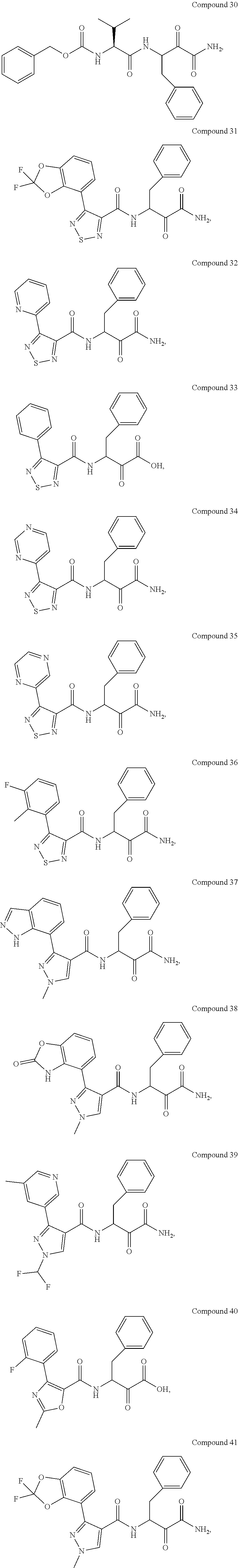

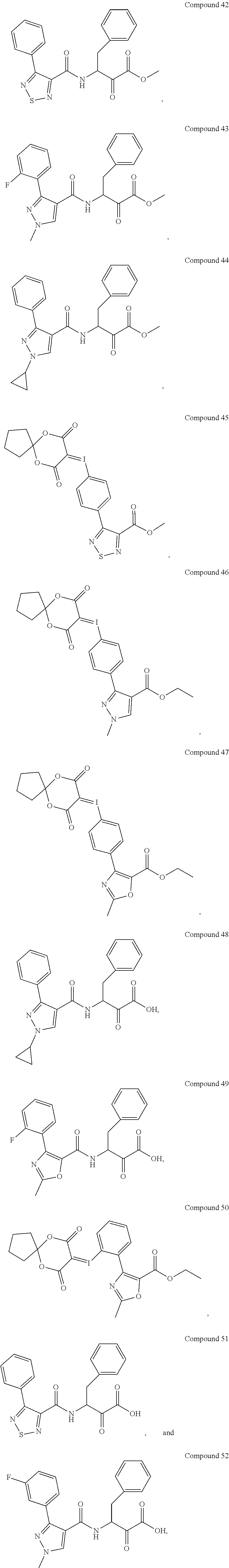

and any combination thereof; or pharmaceutically acceptable salts thereof; and

then, ceasing administration of the compound or administering a second daily amount of the compound for a second number of days, wherein the second daily amount of the compound is less than, greater than, or the same as the first daily amount; and then, administering a third daily amount of the compound for a third number of days to the subject.

In some embodiments according to the methods and compositions of the present disclosure, the first and third daily amounts are the same. In some embodiments, the third daily amount is less than the first daily amount.

In some embodiments, the compound is administered once per week, twice per week, three times per week, or four times per week. In some embodiments, the compound is administered every other day, every third day, every fourth day, every fifth day, or every sixth day.

In some embodiments according to the methods and compositions described herein, the second and third daily amounts of the compound or compounds to be administered are the same. In some embodiments, the third daily amount is greater than the second daily amount.

In some embodiments as described herein, the first and third number of days on which the one or more compounds are administered to the subject are the same. In some embodiments, the first, second, and third number of days are the same. In some embodiments, the third number of days is less than the first number of days. In some embodiments, the first, second, and third number of days are independently selected from 1 to 90, from 1 to 30, from 1 to 20, from 1 to 10, or from 1 to 5. In some embodiments, the first and third number of days is 1 and the second number of days is 1. In some embodiments, the first and third number of days is 1 and the second number of days is 2. In some embodiments, the first and third number of days is 3 and the second number of days is 4. In some embodiments, the first and third number of days is 4 and the second number of days is 3. In some embodiments, the first and third number of days is 4 and the second number of days is 4. In some embodiments, the first and third number of days is 5 and the second number of days is 4. In some embodiments, the first and third number of days is 4 and the second number of days is 5. In some embodiments, the first and third number of days is 10 and the second number of days is 10. In some embodiments, the first and third number of days is 30 and the second number of days is 30. In some embodiments, the first and third number of days is 2 and the second number of days is 1. In some embodiments, the first and third number of days is 30 and the second number of days is 30.

In some embodiments according to the methods and compositions described herein, the frequency of administration of one or more compounds to a subject during the first and third number of days is once per day. In some embodiments, administration of the compound is ceased for the second number of days. In some embodiments, the second daily amount of the compound is administered for the second number of days.

In some embodiments according to the present disclosure, the methods described herein further comprise monitoring the subject's levels of any of said compounds and ceasing administration of said compound or administering the second daily amount of said compound when the level of said compound are above a first threshold value and resuming administration of the compound at the first daily amount when the level of said compound is below a second threshold value. In some embodiments, the first and second threshold values are the same.

In some embodiments according to the methods and compositions described herein, the total weekly dosage of the compound during the first number of days is from 40 to 150 mg. In some embodiments, the total weekly dosage of the compound during the first number of days is from 50 to 90 mg. In some embodiments, the total weekly dosage of the compound during the first number of days is from 60 to 80 mg. In some embodiments, the weekly dosage of the compound during the first number of days is from 5 to 250 mg.

In some embodiments as described herein, the maximum serum concentration of a compound according to the present disclosure during the third number of days is 100 ng/mL or less. In some embodiments, the maximum serum concentration of the compound during the entire treatment period is 100 ng/mL or less.

In some embodiments, the methods of the present disclosure comprise ceasing administration of the compound or administering the second daily amount of the compound for a fourth number of days, then administering the third daily amount of the compound for a fifth number of days; and then repeating said ceasing administration or administering the second daily amount for the fourth number of days, and said administering the third daily amount of the compound for the fifth number of days.

In some embodiments, the disease or condition to be treated comprises a fibrotic condition, which may be one or more of liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases or disorders or any symptom or sequela thereof, or any combination thereof.

### BRIEF DESCRIPTION OF THE DRAWINGS

FIG. 1: Dissociation of Compound 10 from calpain 2 (CAPN2). Complexes of Compound 10 with either CatK or CAPN2 were generated, followed by dilution into substrate-containing assay wells to initiate data collection. Reactivation of the target enzymes due to dissociation of the enzyme-inhibitor complexes was observed as a function of time. CatK shows almost an immediate reactivation following dilution of the CatK-Compound 10 complex suggesting rapid dissociation and recovery of activity (upper solid line, compare to unbound CatK control, lower dashed line). CAPN2 inhibition is retained (lower solid line) relative to unbound. CAPN2 control (upper dashed line). Thus, inhibition of CAPN2 by Compound 10 is retained even in the absence of excess Compound 10. Further, this effect is selective and provides a basis for specific action of Compound 10 against CAPN2.

FIG. 2: Dissociation of Compound 10 from calpain 2 (CAPN2). Complexes of Compound 10 with CAPN2 were generated, followed by dilution into an excess of Compound 29 (a probe for unbound CAPN2). Samples were taken at the time points (lanes 1-6 for 0, 1, 2, 4, 7 and 24 hr respectively) and analyzed by gel electrophoresis to determine the amount of Compound 29 probe incorporation. Lanes 7 and 8 reflect negative and positive controls for probe labeling, respectively. The dissociation rate and half-life for the Compound 10-CAPN2 complex was then calculated.

### DETAILED DESCRIPTION

The present disclosure provides methods for providing to a subject a therapeutically effective amount of the compositions disclosed herein, providing dosing regimens that allow the interaction of said compounds with their targets, including calpain inhibitors including inhibitors of calpain 1 (CAPN1), calpain 2 (CAPN2) or calpain 9 (CAPN9), while also minimizing off-pathway effects, toxicities, and/or side effects that may be associated with the presence of excess unbound drug in the circulation, tissues, organs, cells, fluids, or other bodily matter of a subject. In particular, the present disclosure provides methods for the administration of one or more of the compounds:

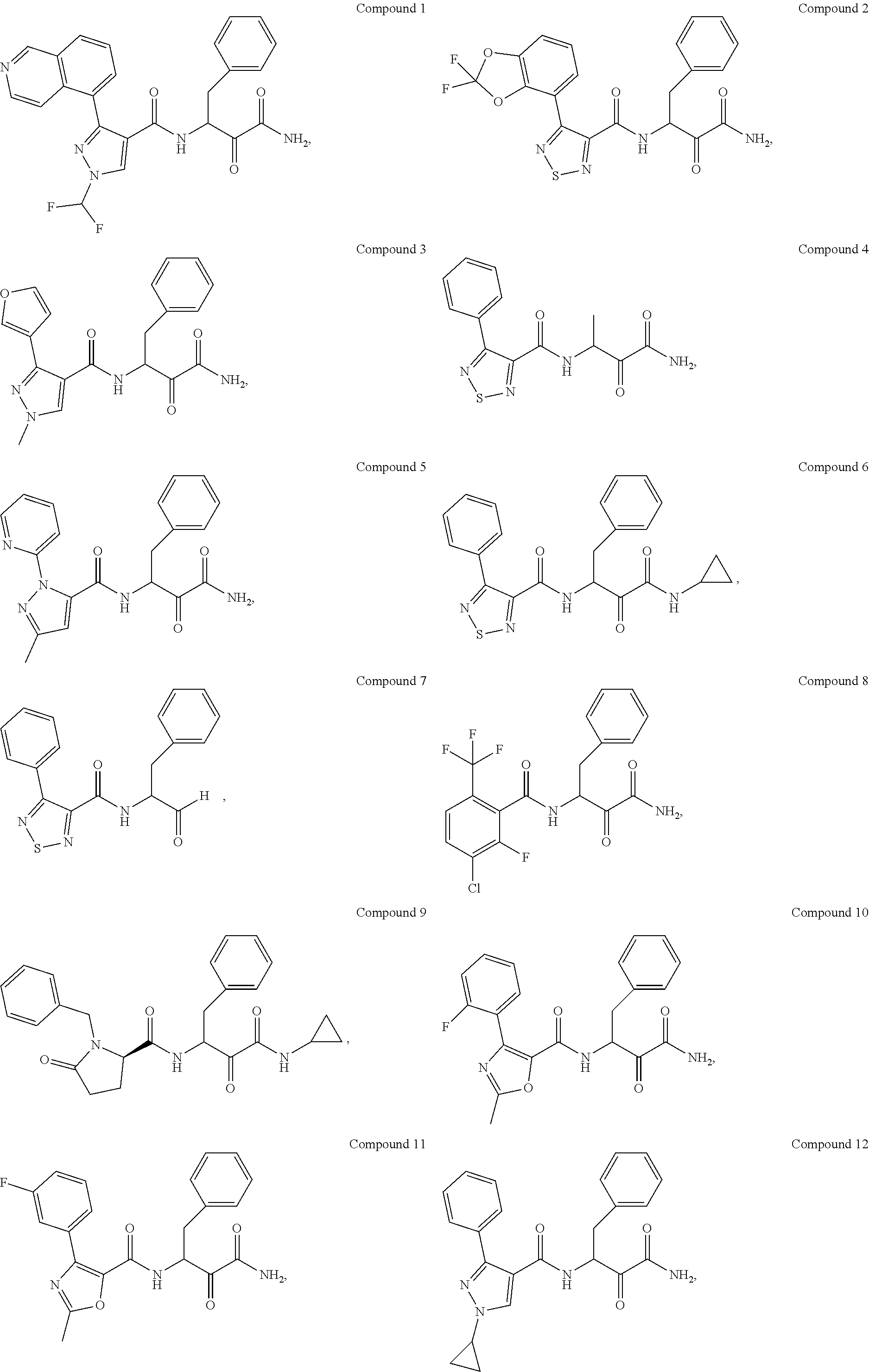

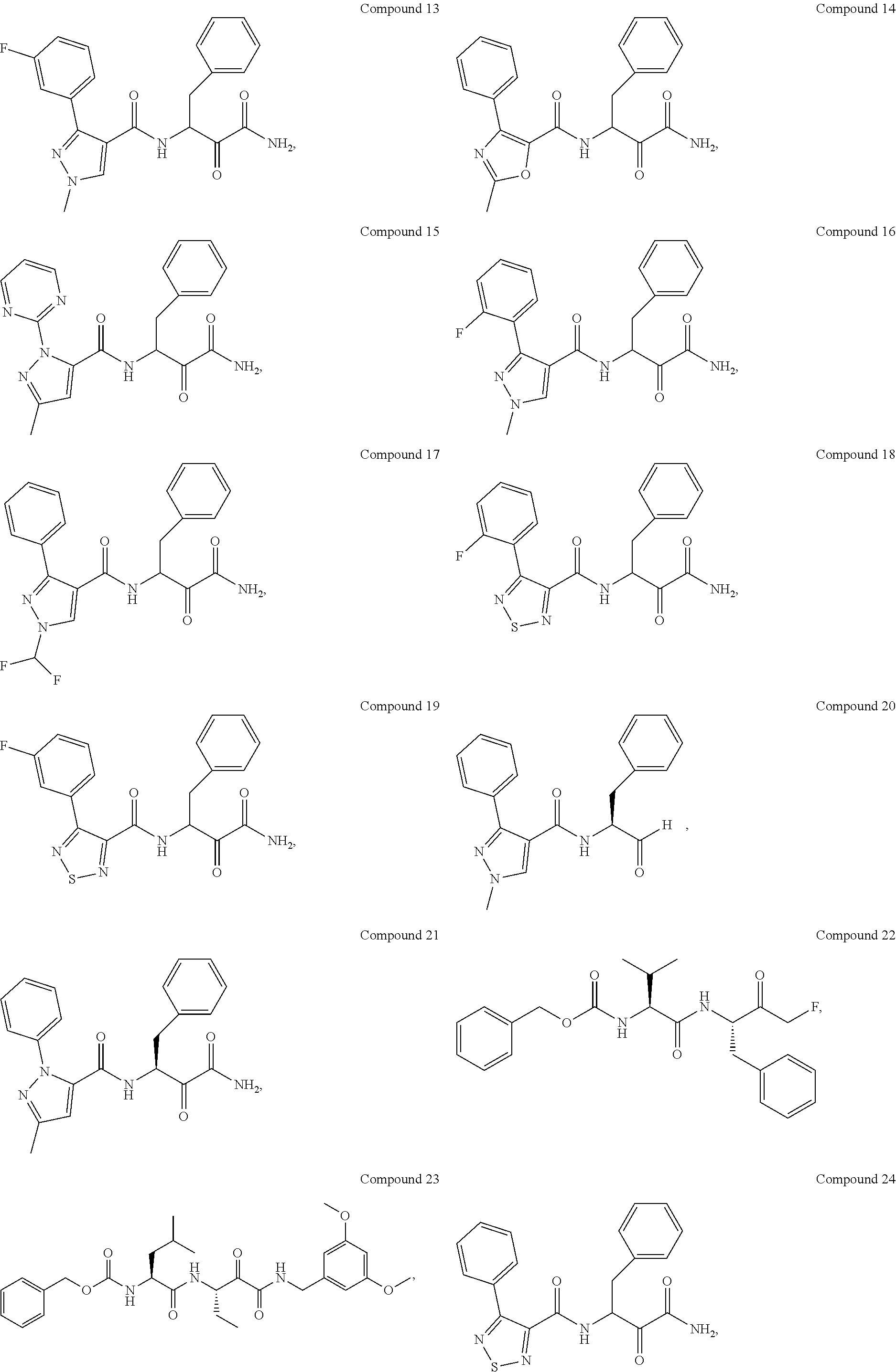

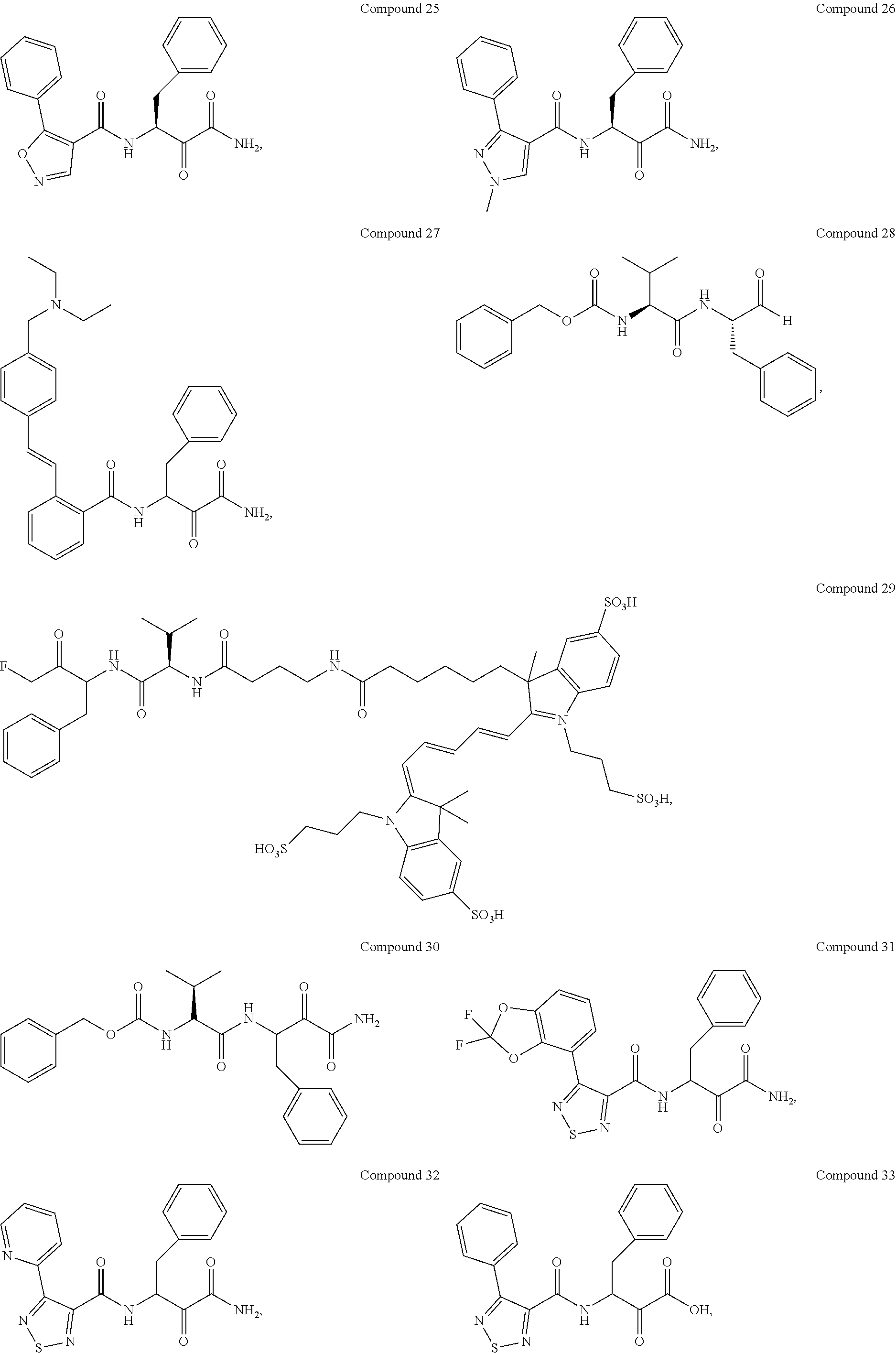

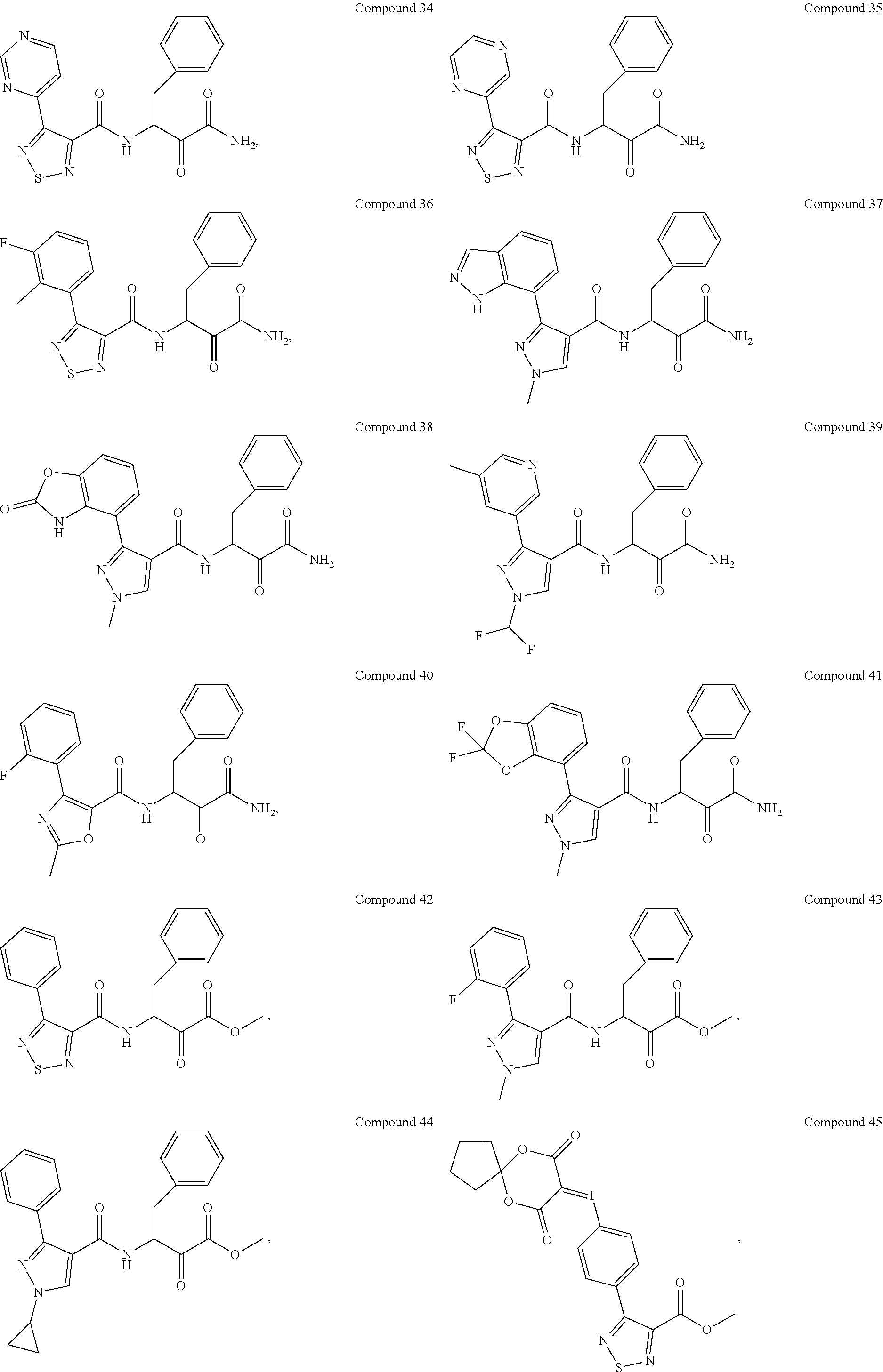

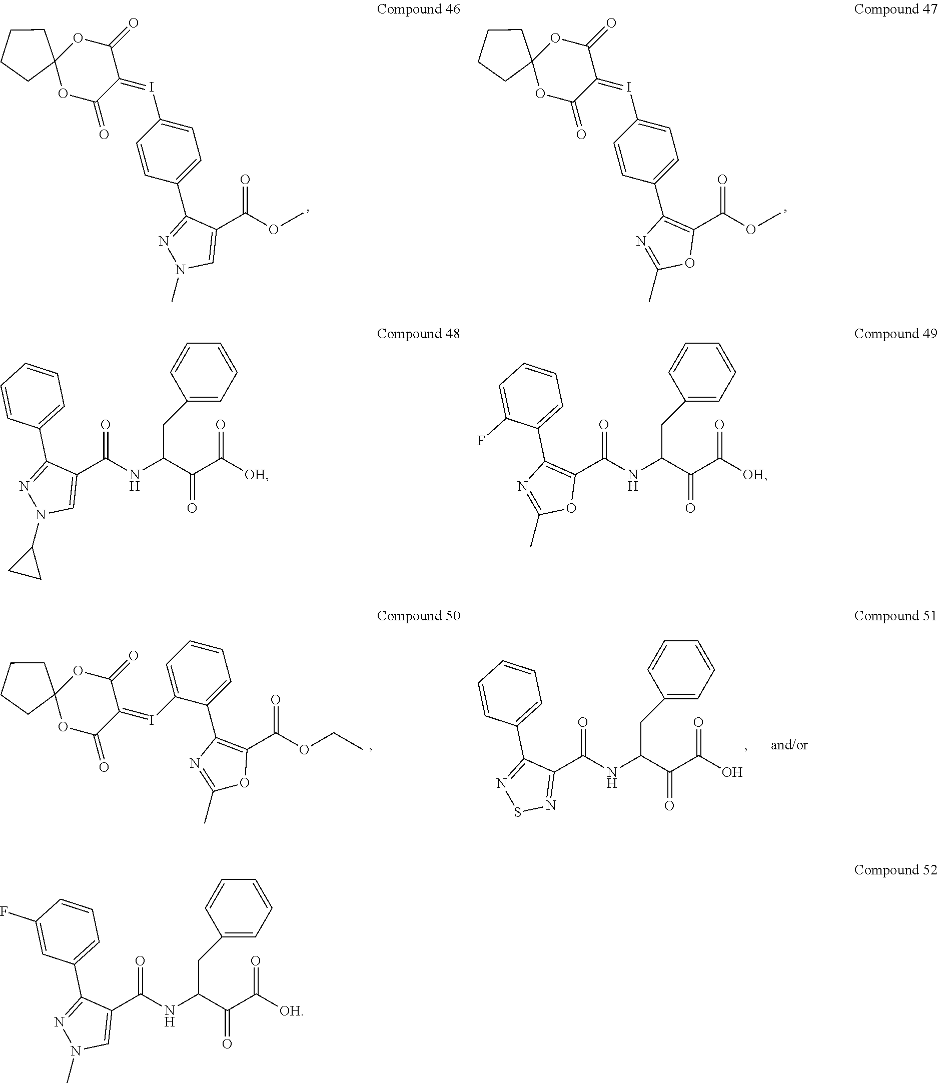

In some embodiments, the present disclosure provides methods for the administration of one or more compounds having the structure of the formula:

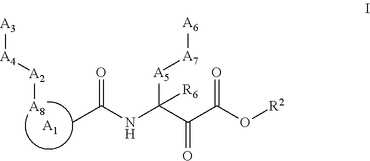

or a pharmaceutically acceptable salt thereof, wherein:

A₁ is selected from the group consisting of optionally substituted. 5-10 membered heterocyclyl; optionally substituted 5-, 8-, or 9-membered heteroaryl; and optionally substituted C₃₋₁₀ carbocyclyl;

A₂ is selected from the group consisting of optionally substituted 3-10 membered heterocyclyl, optionally substituted. C₆₋₁₀ aryl, optionally substituted. 5-10 membered heteroaryl, optionally substituted C₃₋₁₀ carbocyclyl, —CR₂—, —S—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;

when A₂ and A₄ are single bond, A₃ is directly attached to A₈;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted. C₃₋₁₀ carbocyclyl, —C≡CH and optionally substituted 2- to 5-membered polyethylene glycol;

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted alkyl, optionally substituted C₂₋₈ alkenyl, optionally substituted —O—C₁₋₆ alkyl, optionally substituted —OC₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁸ is attached;

A₈ is a ring member of A₁ and is selected from the group consisting of C and N;

R is independently selected from —H, optionally substituted C₁₋₄ alkyl, optionally substituted. C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted. C₆₋₁₀ aryl, optionally substituted. C₆₋₁₀ aryl(C₁-C₆)alkyl, and optionally substituted 5-10 membered heteroaryl;

R² is independently selected from —H, optionally substituted. C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R⁶ is independently selected from —H and optionally substituted C₁₋₄ alkyl; and each n is independently selected to be an integer from 0 to 3.

In some embodiments, the present disclosure provides methods for the administration of one or more compounds having the structure of the formula:

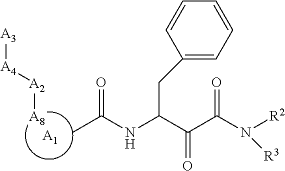

or a pharmaceutically acceptable salt thereof, wherein:

A₁ is selected, from the group consisting of optionally substituted 5-10 membered heterocyclyl provided the 5-10-membered heterocyclyl is not substituted with oxo; optionally substituted 5-, 8-, or 9-membered heteroaryl; and optionally substituted C₃_10 carbocyclyl;

A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —OC(O)NH—, —NHC(O)NH—, —NHC(O)O—, —NHC(O)—, —NHC(S)O—, —NHC(S)—, and single bond;

A₄ is selected from the group consisting of optionally substituted C₆10 aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S(═O)—(CR₂)ₙ—, —(CR₂)ₙ—SO₂—(CR₂)ₙ—, —(CR₂)ₙ—O(═S)—(CR₂)ₙ, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ, —(CR₂)ₙNHC(S)—(CR₂)ₙ, and single bond;

when A₂ and A₄ are single bond, A₃ is directly attached to A₈;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted 03.10 carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;

A₈ is a ring member of A₁ and is selected from the group consisting of C and N;

R is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R² and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and

R⁶ is independently selected from —H and optionally substituted C₁₋₄ alkyl; and each n is independently selected to be an integer from 0 to 3.

In some embodiments of Formula II, A₁ is a 5-membered heteroaryl. In some embodiments of Formula II, A₂ and A₄ are single bonds and A₃ is selected from optionally substituted phenyl and optionally substituted 5-10 membered heteroaryl (for example, optionally substituted 6-membered heteroaryl). The compounds of formulas 1 and II, and Compounds 1-52 may be made according to the methods described in PCT Application No. PCT/US2017/053629 (Publication No. WO2018/064119) and PCT Application No. PCT/US2019/023457, the disclosures of which are incorporated herein by reference in their entirety.

Such administration may be at dosing levels that are reduced, delayed, or altered relative to the levels determined to provide initial efficacy of said compounds. Such administration may further provide that said compounds remain bound to their targets even under conditions in which significantly lower levels of said compounds are present in the circulation or surrounding medium. Such administration may provide that said compounds are present in the circulation or surrounding medium at levels below those that would be predicted by equilibrium models of target binding. Such administration may further provide that said compounds are absent, or substantially absent, in the circulation, bodily fluids, or surrounding medium even while maintaining levels of compound bound to target receptors sufficient to provide clinical effect and/or clinical efficacy.

### Definitions

The term “mammal” is used in its usual biological sense. Thus, it specifically includes humans and non-human mammals such as dogs, cats, horses, donkeys, mules, cows, domestic buffaloes, camels, llamas, alpacas, bison, yaks, goats, sheep, pigs, elk, deer, domestic antelopes, and non-human primates as well as many other species.

“Subject” as used herein, means a human or a non-human mammal including but not limited, to a dog, cat, horse, donkey, mule, cow, domestic buffalo, camel, llama, alpaca, bison, yak, goat, sheep, pig, elk, deer, domestic antelope, or a non-human primate selected, for treatment or therapy.

“Subject suspected of having” means a subject exhibiting one or more clinical indicators of a disease or condition. In certain embodiments, the disease or condition is a fibrotic disease or implicates a fibrotic state. In some embodiments, the disease or condition comprises one or more of liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases or disorders or any symptom or sequela thereof, or any combination thereof in certain embodiments, the disease or condition is associated with CAPN1, CAPN2 or CAPN9.

“Subject in need thereof” means a subject identified as in need of a therapy or treatment.

A therapeutic effect relieves, to some extent, one or more of the symptoms of a disease or disorder, and includes curing the disease or disorder. “Curing” means that the symptoms of active disease are eliminated. However, certain long-team or permanent effects of the disease may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

“Administering” means providing a pharmaceutical agent or composition to a subject, and includes, but is not limited to, administering by a medical professional and self-administering.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

“Parenteral administration,” means administration through injection or infusion. Parenteral administration includes, but is not limited to, subcutaneous administration, intravenous administration, intramuscular administration, intraarterial administration, and intracranial administration.

“Subcutaneous administration” means administration just below the skin.

“Intravenous administration” means administration into a vein.

“Intraarterial administration” means administration into an artery.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent. For example, a pharmaceutical composition may comprise a modified oligonucleotide and a sterile aqueous solution.

“Active pharmaceutical ingredient” means the substance in a pharmaceutical composition that provides a desired effect.

The term “pharmaceutically acceptable salt” refers to salts that retain the biological effectiveness and properties of the compounds with which they are associated and, which are not biologically or otherwise undesirable. In many cases, the compounds herein are capable of forming acid and/or base salts by virtue of the presence of phenol and/or phosphonate groups or groups similar thereto. One of ordinary skill in the art will be aware that the protonation state of any or all of these compounds may vary with pH and ionic character of the surrounding solution, and thus the present disclosure contemplates multiple charge states of each compound. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Solvate” refers to the compound formed by the interaction of a solvent and an EN, a metabolite, or salt thereof. Suitable solvates are pharmaceutically acceptable solvates including hydrates.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein, or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is determined by the way the compound is to be administered.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

The methods as described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and inching agents. Liquid oral dosage forms include aqueous or non-aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

As used herein, a solid dosage loan or “solid form” of an active pharmaceutical ingredient may comprise one or more of a crystalline state, an amorphous state, a glassy state, or any such form as does not consist of said active pharmaceutical ingredient dissolved in a liquid, or any combination thereof. Preferred solid dosage forms include those that are suitable for incorporation into tablets, capsules, sachets, and/or suppositories.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid, microcrystalline cellulose, carboxymethyl cellulose, and talc. Tablets may also comprise solubilizers or emulsifiers, such as poloxamers, cremophor/Kolliphor®/Lutrol®, methylcellulose, hydroxypropylmethylcellulose, or others as are known in the art. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which can be readily made by a person skilled in the art.

Peroral (PO) compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, including for transdermal administration, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA Pharm. Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual unit dose of the active compounds described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 8 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight, or a range between any two of these values. In some embodiments, the actual unit dose is 0.05, 0.07, 0.1, 0.3, 1.0, 3.0, 5.0, 7.0, 10.0 or 25.0 mg/kg of body weight, or a range between any two of these values. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, from about 20 mg to about 300 mg, from about 600 mg to about 1200 mg, or from about 200 mg to about 2000 mg. In some embodiments, the actual unit dose is 5 mg. In some embodiments the actual unit dose is 10 mg. In some embodiments, the actual unit dose is 25 mg. In some embodiments, the actual unit dose is 1500 mg or less. In some embodiments, the actual unit dose is 1250 mg or less. In some embodiments, the actual unit dose is 1000 mg or less. In some embodiments, the actual unit dose is 750 mg or less. In some embodiments, the actual unit dose is 500 mg or less. In some embodiments, the actual unit dose is 250 mg or less.

“Loading dose,” as used herein refers to an initial dose of a compound which is higher than subsequent doses.

“Maintenance dose,” as used herein refers to a subsequent dose that follows a loading dose, and occurs later in time than a loading dose. One of ordinary skill in the art will be aware that the dosage form or mode of administration of a maintenance dose may be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays may be incorporated into the dosing period of the maintenance dose. Such dosing holidays may occur immediately after the administration of the loading dose or at any time during the period of administration of the maintenance dose. As used herein, the period of administration of the maintenance dose may be referred to as the “maintenance phase” of the treatment period.

“Mode of administration” as used herein refers to the means by which a compound is administered to a subject. As used herein, “mode of administration” comprises the dosage form (for example, a tablet, powder, dissolved liquid, suspension, emulsion, aerosol, etc.) and mechanism by which the dosage form is applied to the subject (for example, by injection, such as subcutaneously, intramuscularly, intraperitoneally, intravenously, or intraarterially; topically, such as by cream, lotion, or patch; orally, such as by a pill, dissolved liquid, oral suspension, buccal film, or mouthrinse; nasally, such as by a nasal aerosol, powder, or spray; or ocularly, such as by an eye drop). As used herein, “mode of administration” also comprises the dose, dose amount, and dosing schedule by which a compound is administered to a subject.

In some embodiments, the mode of administration comprises administering a loading dose followed by a maintenance dose. In some embodiments, the loading dose is 2500 mg or less, 2250 mg or less, 2000 mg or less, 1750 mg or less, 1500 mg or less, 1250 mg or less, 1.000 mg or less; 750 mg or less, 500 mg or less, 250 mg or less, 200 mg or less, 150 mg or less, or 100 mg or less, or a range between any two of these values. In some embodiments, the maintenance dose is 300 mg or less; 200 mg or less, 100 mg or less, 50 mg or less, 25 mg or less, 10 mg or less, 5 mg or less, or 1 mg or less, or a range between any two of these values.

In some embodiments the loading dose is administered over a period of one day. In some embodiments the loading dose is administered over a period of 2 days. In some embodiments the loading dose is administered over a period of 3 days. In some embodiments the loading dose is administered over a period of 4 days. In some embodiments the loading dose is administered over a period of 5, 6 or 7 days. In some embodiments, the loading dose is administered over a period of 8-14 days or fewer. In some embodiments, the loading dose is administered over a period of 14 days.

As used herein, “duration of the treatment” refers to the time commencing with administration of the first dose and concluding with the administration of the final dose, such length of time being determined by one of ordinary skill in the art of treating fibrotic diseases or conditions and symptoms and sequelae thereof; and/or diseases or conditions implicating CAPN1, CAPN2 or CAPN9, with reference to the symptoms and health of the subject being treated therefor.

As used herein, “dosing holiday” refers to a period of 24 hours or more during which either no dose is administered to the subject, or a reduced dose is administered to the subject. As used herein, “reduced dose” refers to a dose that is less than the total daily dose to be administered to a subject.

As contemplated herein, enhanced pharmacokinetics or enhanced delivery of the compositions described herein comprises an effect of a treatment method wherein the level of drug bound to the target receptor is substantially the same as that seen in daily dosing at between about 100 and about 400, between about 300 and 600, between about 500 and 1000 mg/day, or between about 750 and 1500 mg/day per subject. In some embodiments, enhanced pharmacokinetics or enhanced delivery of the compositions described herein comprises an effect of a treatment method wherein the level of drug bound to the target receptor is substantially the same as that seen in daily dosing at 1500 mg/day, 1000 mg/day, 800 mg/day, 700 mg/day, 600 mg/day, 500 mg/day, 250 mg/day, or 100 mg/day for an individual subject. In some embodiments, enhanced pharmacokinetics or enhanced delivery of the compositions described herein comprises an effect of a treatment method wherein the level of drug bound to the target receptor is substantially the same as that seen in daily dosing at 100-250 mg/day, 200-500 mg/day, 400-700 mg/day, 500 mg/day, 1000 mg/day, or between about 600 and 1200 mg/day for an individual subject.

Other embodiments disclosed herein include a pharmaceutical composition comprising a therapeutically effective amount of a compound disclosed herein and a pharmaceutically acceptable excipient.

In some embodiments the compositions and methods of the present disclosure provide a method of treating diseases and conditions mediated at least in part by the physiologic effects of CAPN1, CAPN2, or CAPN9, or combinations thereof, comprising administering to a subject in need thereof a compound disclosed herein.

In some embodiments, compounds disclosed herein are specific and/or selective inhibitors of one or more of CAPN1, CAPN2 or CAPN9, or any combination thereof.

In some embodiments, compounds disclosed herein are selective inhibitors of CAPN1 and CAPN2, or CAPN1 and CAPN9, or CAPN2 and CAPN9.

In some embodiments, compounds disclosed herein are effective inhibitors of CAPN1, CAPN2 and/or CAPN9.

In some embodiments, the compounds disclosed herein are broadly effective in treating a host of conditions arising from fibrosis or inflammation, and specifically including those associated with myofibroblast differentiation. Accordingly, compounds disclosed herein are active therapeutics for a diverse set of diseases or disorders that include or that produces a symptom which include, but are not limited to: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases or disorders. In other embodiments, the compounds disclosed herein can be used can be used in metabolic and reaction kinetic studies, detection and imaging techniques and radioactive treatments.

In some embodiments, the compounds disclosed herein are used to treat diseases or conditions or that produces a symptom in a subject which include, but not limited to: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases.

In certain embodiments methods are provided for alleviating or ameliorating a condition or disorder, affected at least in part by the enzymatic activity of calpain 1 (CAPN1), calpain 2 (CAPN2), and/or calpain 9 (CAPN9), or mediated at least in part by the enzymatic activity of CAPN1, CAPN2, and/or CAPN9 wherein the condition includes or produces a symptom which includes: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and/or rheumatoid arthritis.

In some embodiments, the methods, compounds, and/or compositions of the present invention are used for prophylactic therapy.

In some embodiments, the CAPN1, CAPN2, and/or CAPN9 inhibiting compounds demonstrate efficacy in animal models of human disease. Specifically, in-vivo treatment of mice, rabbits, and other mammalian subjects with compounds disclosed herein establish the utility of these compounds as therapeutic agents to modulate CAPN1, CAPN2, and/or CAPN9 activities in humans and thereby ameliorate corresponding medical conditions.

Some embodiments provide compounds, pharmaceutical compositions, and methods of use to inhibit myofibroblast differentiation. Some embodiments provide compounds, pharmaceutical compositions, and methods of use for inhibiting CAPN1, CAPN2, and/or CAPN9 or combinations of these enzyme activities such as CAPN1 and CAPN2, or CAPN1 and CAPN9, or CAPN2 and CAPN9. Some embodiments provide methods for treatment of diseases and disorders by inhibiting CAPN1, CAPN2, and/or CAPN9 or combinations of these enzymatic activities.

In previous trials, administration of these compounds has been shown to be effective in animal models for the inhibition of CAPN1, CAPN2, and/or CAPN9. See e.g., International Application No. PCT/US2017/053629, which is hereby incorporated by reference in its entirety. Therefore it is the object of this disclosure to describe methods of administering the compositions as described herein in such a way as to retain their primary effect as modulators of CAPN1, CAPN2, and/or CAPN9 activity, and the relief of clinical symptoms achieved thereby, utilizing reduced dosages which may provide for enhanced patient compliance with dosing instructions, as well as reduced incidence of off-pathway effects, toxicities, and side effects related to the presence of free (unbound) drug in the circulation or otherwise partitioned into the tissues, organs, or bodily matter of a subject.

Without intending to be bound by any particular theory, the compounds of the present disclosure have been found to bind specifically to their target receptors, with low off-rates, allowing longer duration between doses and/or the opportunity for dosing regimens in which the amount of free compound in the patient's circulation, cells, tissues, or other bodily matter is allowed to fall, even to the point at which said free compound is effectively “washed out” of the patient. In such a scenario, the compounds as disclosed herein remain bound to their target receptors and thus maintain clinical efficacy even while the amount of free compound can be reduced to below the level at which off-pathway effects, side effects, or toxicities may be triggered. In some embodiments, the target receptors comprise one or more of CAPN1, CAPN2, and/or CAPN9. In some embodiments, the clinical or therapeutic efficacy that is retained results in the treatment, amelioration, prevention, or cure of one or more fibrotic conditions. In some embodiments, said fibrotic condition may comprise one or more of liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases or disorders or any symptom or sequela thereof or any combination thereof.

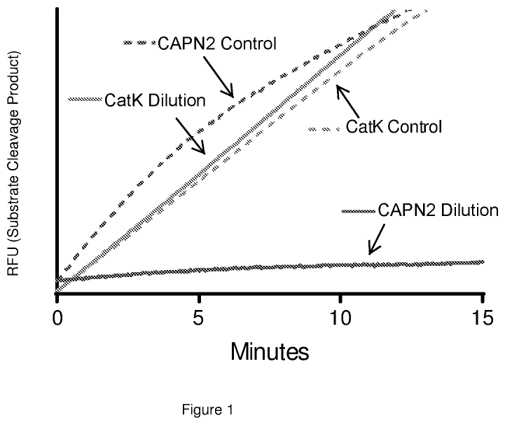

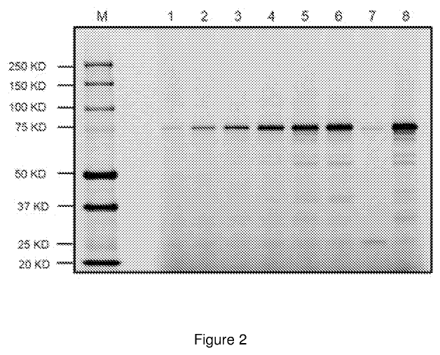

In some embodiments, the compounds of the present disclosure bind irreversibly to their target, which may provide an unexpectedly high therapeutic efficacy based on a long-lasting and highly specific interaction. In some embodiments, the compounds of the present disclosure form complexes with their physiological target having half lives in vitro ranging from 5-600 minutes. In some embodiments, the compounds of the present disclosure form complexes with their physiological target ranging from 5-20 minutes, from 10-50 minutes, from 20-100 minutes, from 50-250 minutes, from 100-300 minutes, from 150-400 minutes, from 200-500 minutes, from 300-60 minutes, or any value within said ranges, or any range comprising any of the values described herein. In some embodiments, the compounds of the present disclosure form complexes with their physiological targets that are irreversible and/or nondissociable under physiological conditions. The half-lives of the compounds of the present disclosure have been determined according to methods illustrated in FIG. 1 and FIG. 2 and in Example 1.

According to the methods disclosed herein, reduction of side effects related to administration of the above-mentioned compounds may be achieved by modulating the dosing schedule such that subjects experience periodic partial or full reductions in dosing for fixed amounts of time. In some embodiments, said periodic partial or full reduction in dosing is followed by a partial or full resumption of dosing. In some embodiments, dosages are administered daily for between one and thirty days, followed by a dosing holiday lasting for between one and thirty days. In some embodiments, during the dosing holiday, no dose is administered. In some further embodiments, the compound and its metabolites are allowed to clear completely from the subject's body prior to administration of the next dose. In some other embodiments, during the dosing holiday, a dose less than the usual daily dose is administered. In some further embodiments, an amount of the administered compound less than the therapeutically effective amount is allowed to remain within the subject during the dosing holiday. In some further embodiments, an amount of the administered compound sufficient to maintain therapeutic levels in the affected tissues is allowed to remain within the subject.

In some embodiments, the maximum serum concentration (C_{Max}) of the compound during the dosing schedule is less than 20 ug/ml, less than 15 ug/ml, less than 13 ug/ml, less than 10 ug/ml, less than 5 ug/ml, less than 3 ug/ml, less than 1 ug/ml, less than 800 ng/ml, less than 500 ng/ml, less than 120 ng/ml, less than 100 ng/ml, less than 90 ng/ml, less than 80 ng/ml, less than 70 ng/ml, less than 60 ng/ml, or less than 50 ng/ml, or a range between any two of these values. In some embodiments, the minimum serum concentration during the dosing schedule is less than 10 ng/ml, less than 1 ng/ml, less than 0.1 ng/ml, less than 0.01 ng/ml, or less than 0.001 ng/ml, or a range between any two of these values. In some embodiments, the level of the compound administered during the dosing schedule may be undetectable during some portion of the dosing holiday.

In some embodiments, the maximum serum concentration of the compound during the dosing schedule is higher during an initial phase of administration, and lower in subsequent phases. In some embodiments, the maximum serum concentration of the compound during the initial (loading) phase of administration is less than 20 ug/ml, less than 15 ug/ml, less than 13 ug/ml, less than 10 ug/ml, less than 5 ug/ml, less than 3 ug/ml, less than 1 ug/ml, less than 800 ng/ml, less than 500 ng/ml, less than 400 ng/ml, less than 300 ng/ml, less than 200 ng/ml, less than 150 ng/ml, less than 120 ng/ml, less than 100 ng/ml, less than 90 ng/ml, less than 80 ng/ml, less than 70 ng/ml, less than 60 ng/ml, or less than 50 ng/ml, or a range between any two of these values. In some such embodiments, the maximum serum concentration during the initial phase of administration is from 5 ng/ml to 250 ng/ml. In some such embodiments, the maximum serum concentration during the initial phase of administration is from 200 ng/ml to 2 ug/ml. In some such embodiments, the maximum serum concentration during the initial phase of administration is from 1 ug/ml to 20 ug/ml. In some embodiments, the maximum serum concentration of the compound during the subsequent (maintenance) phase of administration is less than 20 ug/ml, less than 15 ug/ml, less than 13 ug/ml, less than 10 ug/ml, less than 5 ug/ml, less than 3 ug/ml, less than 1 ug/ml, less than 800 ng/ml, less than 500 ng/ml, less than 350 ng/ml, less than 200 ng/ml, less than 120 ng/ml, less than 100 ng/ml, less than 90 ng/ml, less than 80 ng/ml, less than 70 ng/ml, less than 60 ng/ml, or less than 50 ng/ml, less than 40 ng/ml, less than 35 ng/ml, or less than 10 ng/ml, or a range between any two of these values. One of ordinary skill in the art will readily be aware of such methods as exist in the art for the monitoring of serum concentrations of pharmaceutical agents, and means of adjusting dosages of the compounds disclosed herein in order to achieve the desired serum concentrations.

In some embodiments, the maximum serum concentration of compounds of the present disclosure during the initial (loading) phase of administration is 20 ug/ml or less, 15 ug/ml or less, 13 ug/ml or less, 10 ug/ml or less, 5 ug/ml or less, 3 ug/ml or less, 1 ug/ml or less, 800 ng/ml or less, 500 ng/ml or less, 450 ng/ml or less, 400 ng/ml or less, 350 ng/ml or less, 300 ng/ml or less, or 250 ng/ml or less, or a range between any two of these values. In some embodiments, the maximum serum concentration of the compounds disclosed herein during the subsequent (maintenance) phase of administration is 20 ug/ml or less, 15 ug/ml or less, 13 ug/ml or less, 10 ug/ml or less, 5 ug/ml or less, 3 ug/ml or less, 1 ug/ml or less, 800 ng/ml or less, 500 ng/ml or less, 450 ng/ml or less, 400 ng/ml or less, 350 rig/ml or less, 300 ng/ml or less, 250 ng/ml or less, 200 ng/ml or less, 150 ng/ml or less, or 120 ng/ml or less, or a range between any two of these values.

According to the present disclosure, the dosing schedule may be varied in order to attain the desired therapeutic effect while eliminating side effects, toxicities, or off-pathway effects. In each of the following embodiments, variations in dosing schedule as described may be repeated throughout the duration of the treatment. In each of the following embodiments, the first dosage may be higher, lower, or the same as the dosages following the first dosage. In each of the following embodiments, a loading dose may precede the disclosed dosing regimen, and a dosing holiday may or may not follow the administration of the loading dose.

In some embodiments, dosages are administered daily, every other day, every third day, every fourth day, every fifth day, or every sixth day. In some embodiments, dosages are administered weekly. In some embodiments, dosages are administered more often than weekly, such as twice per week, three times per week, four times per week, five times per week, or six times per week. In some embodiments, dosages are administered monthly, or more often than monthly, such as 2, 3, 4, 5, 6, 7, 8, 9, 10, 11, 12, 13, 14, 15, 16, 17, 18, 19, 20, 21, 22, 23, 24, 25, 26, 27, 28, 29, or 30 times per month.

In some embodiments, dosages are administered every other day for the duration of the treatment. In other embodiments, dosages are administered on two out of every three days for the duration of the treatment. In still other embodiments, dosages are administered two out of every four days for the duration of the treatment. In some embodiments, dosages are administered daily for one day, followed by a two day dosing holiday. In some embodiments, dosages are administered daily for one day, followed by a two day dosing holiday. In some embodiments, dosages are administered daily for one day, followed by a three day dosing holiday. In some embodiments, dosages are administered daily for one day, followed by a four day dosing holiday. In some embodiments, dosages are administered daily for one day, followed by a five day dosing holiday. In some embodiments, dosages are administered daily for one day, followed by a six day dosing holiday. In some embodiments, dosages are administered daily for one day, followed by a seven day dosing holiday. In some embodiments, dosages are administered daily for one day, followed by an eight day dosing holiday. In some embodiments, dosages are administered daily for one day, followed by a nine day dosing holiday. In some embodiments, dosages are administered daily for one day, followed by a ten day dosing holiday. In some embodiments, dosages are administered daily for one day, followed by an eleven day dosing holiday. In some embodiments, dosages are administered daily for one day, followed by a twelve day dosing holiday. In some embodiments, dosages are administered daily for one day, followed by a thirteen day dosing holiday. In some embodiments, dosages are administered daily for one day, followed by a fourteen day dosing holiday.

In some embodiments, dosages are administered daily for two days, followed, by a one day dosing holiday. In some embodiments, dosages are administered daily for two days, followed by a two day dosing holiday. In some embodiments, dosages are administered daily for two days, followed, by a three day dosing holiday. In some embodiments, dosages are administered daily for two days, followed by a four day dosing holiday. In some embodiments, dosages are administered daily for two days, followed by a five day dosing holiday. In some embodiments, dosages are administered daily for two days, followed by a six day dosing holiday. In some embodiments, dosages are administered daily for two days, followed by a seven day dosing holiday. In some embodiments, dosages are administered daily for two days, followed by an eight day dosing holiday. In some embodiments, dosages are administered daily for two days, followed by a nine day dosing holiday. In some embodiments, dosages are administered daily for two days, followed by a ten day dosing holiday. In some embodiments, dosages are administered daily for two days, followed, by an eleven day dosing holiday. In some embodiments, dosages are administered daily for two days, followed by a twelve day dosing holiday. In some embodiments, dosages are administered daily for two days, followed by a thirteen day dosing holiday. In some embodiments, dosages are administered daily for two days, followed by a fourteen day dosing holiday.

In some embodiments, dosages are administered daily for three days, followed by a one day dosing holiday. In some embodiments, dosages are administered daily for three days, followed by a two day dosing holiday. In some embodiments, dosages are administered daily for three days, followed by a three day dosing holiday. In some embodiments, dosages are administered daily for three days, followed by a four day dosing holiday. In some embodiments, dosages are administered daily for three days, followed by a five day dosing holiday. In some embodiments, dosages are administered, daily for three days, followed by a six day dosing holiday. In some embodiments, dosages are administered daily for three days, followed by a seven day dosing holiday. In some embodiments, dosages are administered daily for three days, followed by an eight day dosing holiday. In some embodiments, dosages are administered daily for three days, followed by a nine day dosing holiday. In some embodiments, dosages are administered daily for three days, followed by a ten day dosing holiday. In some embodiments, dosages are administered daily for three days, followed by an eleven day dosing holiday. In some embodiments, dosages are administered daily for three days, followed by a twelve day dosing holiday. In some embodiments, dosages are administered daily for three days, followed by a thirteen day dosing holiday. In some embodiments, dosages are administered daily for three days, followed by a fourteen day dosing holiday.

In some embodiments, dosages are administered daily for four days, followed by a one day dosing holiday. In some embodiments, dosages are administered daily for four days, followed by a two day dosing holiday. In some embodiments, dosages are administered daily for four days, followed by a three day dosing holiday. In some embodiments, dosages are administered daily for four days, followed by a four day dosing holiday. In some embodiments, dosages are administered daily for four days, followed by a five day dosing holiday. In some embodiments, dosages are administered daily for four days, followed by a six day dosing holiday. In some embodiments, dosages are administered daily for four days, followed by a seven day dosing holiday. In some embodiments, dosages are administered daily for four days, followed by an eight day dosing holiday. In some embodiments, dosages are administered daily for four days, followed by a nine day dosing holiday. In some embodiments, dosages are administered daily for four days, followed by a ten day dosing holiday. In some embodiments, dosages are administered daily for four days, followed by an eleven day dosing holiday. In some embodiments, dosages are administered daily for four days, followed by a twelve day dosing holiday. In some embodiments, dosages are administered daily for four days, followed by a thirteen day dosing holiday. In some embodiments, dosages are administered daily for four days, followed by a fourteen day dosing holiday.

In some embodiments, dosages are administered daily for five days, followed by a one day dosing holiday. In some embodiments, dosages are administered daily for five days, followed by a two day dosing holiday. In some embodiments, dosages are administered daily for five days, followed by a three day dosing holiday. In some embodiments, dosages are administered daily for five days, followed by a four day dosing holiday. In some embodiments, dosages are administered daily for five days, followed by a five day dosing holiday. In some embodiments, dosages are administered daily for five days, followed by a six day dosing holiday. In some embodiments, dosages are administered daily for five days, followed by a seven day dosing holiday. In some embodiments, dosages are administered daily for five days, followed by an eight day dosing holiday. In some embodiments, dosages are administered daily for five days, followed by a nine day dosing holiday. In some embodiments, dosages are administered daily for five days, followed by a ten day dosing holiday. In some embodiments, dosages are administered daily for five days, followed by an eleven day dosing holiday. In some embodiments, dosages are administered daily for five days, followed by a twelve day dosing holiday. In some embodiments, dosages are administered daily for five days, followed by a thirteen day dosing holiday. In some embodiments, dosages are administered daily for five days, followed by a fourteen day dosing holiday.

In some embodiments, dosages are administered daily for six days, followed, by a one day dosing holiday. In some embodiments, dosages are administered daily for six days, followed by a two day dosing holiday. In some embodiments, dosages are administered daily for six days, followed by a three day dosing holiday. In some embodiments, dosages are administered daily for six days, followed by a four day dosing holiday. In some embodiments, dosages are administered daily for six days, followed by a five day dosing holiday. In some embodiments, dosages are administered daily for six days, followed by a six day dosing holiday. In some embodiments, dosages are administered daily for six days, followed by a seven day dosing holiday. In some embodiments, dosages are administered daily for six days, followed by an eight day dosing holiday. In some embodiments, dosages are administered daily for six days, followed by a nine day dosing holiday. In some embodiments, dosages are administered daily for six days, followed by a ten day dosing holiday. In some embodiments, dosages are administered daily for six days, followed by an eleven day dosing holiday. In some embodiments, dosages are administered daily for six days, followed by a twelve day dosing holiday. In some embodiments, dosages are administered daily for six days, followed by a thirteen day dosing holiday. In some embodiments, dosages are administered daily for six days, followed by a fourteen day dosing holiday.

In some embodiments, dosages are administered daily for seven days, followed by a one day dosing holiday. In some embodiments, dosages are administered daily for seven days, followed by a two day dosing holiday. In some embodiments, dosages are administered daily for seven days, followed by a three day dosing holiday. In some embodiments, dosages are administered daily for seven days, followed by a four day dosing holiday. In some embodiments, dosages are administered daily for seven days, followed by a five day dosing holiday. In some embodiments, dosages are administered daily for seven days, followed by a six day dosing holiday. In some embodiments, dosages are administered daily for seven days, followed by a seven day dosing holiday. In some embodiments, dosages are administered daily for seven days, followed by an eight day dosing holiday. In some embodiments, dosages are administered daily for seven days, followed by a nine day dosing holiday. In some embodiments, dosages are administered daily for seven days, followed by a ten day dosing holiday. In some embodiments, dosages are administered daily for seven days, followed by an eleven day dosing holiday. In some embodiments, dosages are administered daily for seven days, followed by a twelve day dosing holiday. In some embodiments, dosages are administered daily for seven days, followed by a thirteen day dosing holiday. In some embodiments, dosages are administered daily for seven days, followed by a fourteen day dosing holiday.

In some embodiments, dosages are administered daily for eight days, followed by a one day dosing holiday. In some embodiments, dosages are administered daily for eight days, followed by a two day dosing holiday. In some embodiments, dosages are administered daily for eight days, followed by a three day dosing holiday. In some embodiments, dosages are administered daily for eight days, followed by a four day dosing holiday. In some embodiments, dosages are administered daily for eight days, followed by a five day dosing holiday. In some embodiments, dosages are administered daily for eight days, followed by a six day dosing holiday. In some embodiments, dosages are administered daily for eight days, followed by a seven day dosing holiday. In some embodiments, dosages are administered daily for eight days, followed by an eight day dosing holiday. In some embodiments, dosages are administered daily for eight days, followed by a nine day dosing holiday. In some embodiments, dosages are administered daily for eight days, followed by a ten day dosing holiday. In some embodiments, dosages are administered daily for eight days, followed by an eleven day dosing holiday. In some embodiments, dosages are administered daily for eight days, followed by a twelve day dosing holiday. In some embodiments, dosages are administered daily for eight days, followed by a thirteen day dosing holiday. In some embodiments, dosages are administered daily for eight days, followed by a fourteen day dosing holiday.

In some embodiments, dosages are administered daily for nine days, followed by a one day dosing holiday. In some embodiments, dosages are administered daily for nine days, followed by a two day dosing holiday. In some embodiments, dosages are administered daily for nine days, followed by a three day dosing holiday. In some embodiments, dosages are administered daily for nine days, followed by a four day dosing holiday. In some embodiments, dosages are administered daily for nine days, followed by a five day dosing holiday. In some embodiments, dosages are administered daily for nine days, followed by a six day dosing holiday. In some embodiments, dosages are administered daily for nine days, followed by a seven day dosing holiday. In some embodiments, dosages are administered daily for nine days, followed by an eight day dosing holiday. In some embodiments, dosages are administered daily for nine days, followed by a nine day dosing holiday. In some embodiments, dosages are administered daily for nine days, followed by a ten day dosing holiday. In some embodiments, dosages are administered daily for nine days, followed by an eleven day dosing holiday. In some embodiments, dosages are administered daily for nine days, followed by a twelve day dosing holiday. In some embodiments, dosages are administered daily for nine days, followed by a thirteen day dosing holiday. In some embodiments, dosages are administered daily for nine days, followed by a fourteen day dosing holiday.

In some embodiments, dosages are administered daily for ten days, followed by a one day dosing holiday. In some embodiments, dosages are administered daily for ten days, followed by a two day dosing holiday. In some embodiments, dosages are administered daily for ten days, followed by a three day dosing holiday. In some embodiments, dosages are administered daily for ten days, followed by a four day dosing holiday. In some embodiments, dosages are administered daily for ten days, followed by a five day dosing holiday. In some embodiments, dosages are administered daily for ten days, followed by a six day dosing holiday. In some embodiments, dosages are administered daily for ten days, followed by a seven day dosing holiday. In some embodiments, dosages are administered daily for ten days, followed by an eight day dosing holiday. In some embodiments, dosages are administered daily for ten days, followed by a nine day dosing holiday. In some embodiments, dosages are administered daily for ten days, followed by a ten day dosing holiday. In some embodiments, dosages are administered daily for ten days, followed by an eleven day dosing holiday. In some embodiments, dosages are administered daily for ten days, followed by a twelve day dosing holiday. In some embodiments, dosages are administered daily for ten days, followed by a thirteen day dosing holiday. In some embodiments, dosages are administered daily for ten days, followed by a fourteen day dosing holiday.

In some embodiments, dosages are administered daily for eleven days, followed, by a one day dosing holiday. In some embodiments, dosages are administered daily for eleven days, followed by a two day dosing holiday. In some embodiments, dosages are administered daily for eleven days, followed by a three day dosing holiday. In some embodiments, dosages are administered daily for eleven days, followed by a four day dosing holiday. In some embodiments, dosages are administered daily for eleven days, followed by a five day dosing holiday. In some embodiments, dosages are administered daily for eleven days, followed by a six day dosing holiday. In some embodiments, dosages are administered daily for eleven days, followed by a seven day dosing holiday. In some embodiments, dosages are administered daily for eleven days, followed by an eight day dosing holiday. In some embodiments, dosages are administered daily for eleven days, followed by a nine day dosing holiday. In some embodiments, dosages are administered daily for eleven days, followed by a ten day dosing holiday. In some embodiments, dosages are administered daily for eleven days, followed by an eleven day dosing holiday. In some embodiments, dosages are administered daily for eleven days, followed by a twelve day dosing holiday. In some embodiments, dosages are administered daily for eleven days, followed by a thirteen day dosing holiday. In some embodiments, dosages are administered daily for eleven days, followed by a fourteen day dosing holiday.

In some embodiments, dosages are administered daily for twelve days, followed by a one day dosing holiday. In some embodiments, dosages are administered daily for twelve days, followed by a two day dosing holiday. In some embodiments, dosages are administered daily for twelve days, followed by a three day dosing holiday. In some embodiments, dosages are administered daily for twelve days, followed by a four day dosing holiday. In some embodiments, dosages are administered daily for twelve days, followed by a five day dosing holiday. In some embodiments, dosages are administered daily for twelve days, followed by a six day dosing holiday. In some embodiments, dosages are administered daily for twelve days, followed by a seven day dosing holiday. In some embodiments, dosages are administered daily for twelve days, followed by an eight day dosing holiday. In some embodiments, dosages are administered daily for twelve days, followed by a nine day dosing holiday. In some embodiments, dosages are administered daily for twelve days, followed by a ten day dosing holiday. In some embodiments, dosages are administered daily for twelve days, followed by an eleven day dosing holiday. In some embodiments, dosages are administered daily for twelve days, followed by a twelve day dosing, holiday. In some embodiments, dosages are administered daily for twelve days, followed by a thirteen day dosing holiday. In some embodiments, dosages are administered daily for twelve days, followed by a fourteen day dosing holiday.

In some embodiments, dosages are administered daily for thirteen days, followed by a one day dosing holiday. In some embodiments, dosages are administered daily for thirteen days, followed by a two day dosing holiday. In some embodiments, dosages are administered daily for thirteen days, followed by a three day dosing holiday. In some embodiments, dosages are administered daily for thirteen days, followed by a four day dosing holiday. In some embodiments, dosages are administered daily for thirteen days, followed by a five day dosing holiday. In some embodiments, dosages are administered daily for thirteen days, followed by a six day dosing holiday. In some embodiments, dosages are administered daily for thirteen days, followed by a seven day dosing holiday. In some embodiments, dosages are administered daily for thirteen days, followed by an eight day dosing holiday. In some embodiments, dosages are administered daily for thirteen days, followed by a nine day dosing holiday. In some embodiments, dosages are administered daily for thirteen days, followed by a ten day dosing holiday. In some embodiments, dosages are administered daily for thirteen days, followed by an eleven day dosing holiday. In some embodiments, dosages are administered daily for thirteen days, followed by a twelve day dosing holiday. In some embodiments, dosages are administered daily for thirteen days, followed by a thirteen day dosing holiday. In some embodiments, dosages are administered daily for thirteen days, followed by a fourteen day dosing holiday.

In some embodiments, dosages are administered daily for fourteen days, followed by a one day dosing holiday. In some embodiments, dosages are administered daily for fourteen days, followed by a two day dosing holiday. In some embodiments, dosages are administered daily for fourteen days, followed by a three day dosing holiday. In some embodiments, dosages are administered daily for fourteen days, followed by a four day dosing holiday. In some embodiments, dosages are administered daily for fourteen days, followed by a five day dosing holiday. In some embodiments, dosages are administered daily for fourteen days, followed by a six day dosing holiday. In some embodiments, dosages are administered daily for fourteen days, followed by a seven day dosing holiday. In some embodiments, dosages are administered daily for fourteen days, followed by an eight day dosing holiday. In some embodiments, dosages are administered daily for fourteen days, followed, by a nine day dosing holiday. In some embodiments, dosages are administered daily for fourteen days, followed by a ten day dosing holiday. In some embodiments, dosages are administered daily for fourteen days, followed by an eleven day dosing holiday. In some embodiments, dosages are administered daily for fourteen days, followed by a twelve day, dosing holiday. In some embodiments, dosages are administered daily for fourteen days, followed by a thirteen day dosing holiday. In some embodiments, dosages are administered daily for fourteen days, followed by a fourteen day dosing holiday.

In some embodiments, dosages are administered daily for thirty days followed by a thirty day dosing holiday. In some embodiments, dosages are administered daily for thirty days followed by a 25-30 day dosing holiday. In some embodiments, dosages are administered daily for thirty days followed by a 20-25 day dosing holiday. In some embodiments, dosages are administered daily for thirty days followed by a 15-20 day dosing holiday. In some embodiments, dosages are administered daily for thirty days followed by a 10-15 day dosing holiday. In some embodiments, dosages are administered daily for thirty days followed by a 5-10 day dosing holiday. In some embodiments, dosages are administered daily for thirty days followed by a 1-5 day dosing holiday.

In some embodiments, dosages are administered daily for 25-30 days followed by a thirty day dosing holiday. In some embodiments, dosages are administered daily for 25-30 days followed by a 25-30 day dosing holiday. In some embodiments, dosages are administered daily for 25-30 days followed by a 20-25 day dosing holiday. In some embodiments, dosages are administered daily for 25-30 days followed by a 15-20 day dosing holiday. In some embodiments, dosages are administered daily for 25-30 days followed by a 10-15 dosing holiday. In some embodiments, dosages are administered daily for 25-30 days followed by a 5-10 day dosing holiday. In some embodiments, dosages are administered daily for 25-30 days followed by a 1-5 day dosing holiday.

In some embodiments, dosages are administered daily for 20-25 days followed by a thirty day dosing holiday. In some embodiments, dosages are administered daily for 20-25 days followed by a 25-30 day dosing holiday. In some embodiments, dosages are administered daily for 20-25 days followed by a 20-25 day dosing holiday. In some embodiments, dosages are administered daily for 20-25 days followed by a 15-20 day dosing holiday. In some embodiments, dosages are administered daily for 20-25 days followed by a 10-15 dosing holiday. In some embodiments, dosages are administered daily for 20-25 days followed by a 5-10 day dosing holiday. In some embodiments, dosages are administered daily for 20-25 days followed by a 1-5 day dosing holiday.

In some embodiments, dosages are administered daily for 15-20 days followed by a thirty day dosing holiday. In some embodiments, dosages are administered daily for 15-20 days followed by a 25-30 day dosing holiday. In some embodiments, dosages are administered daily for 15-20 days followed by a 20-25 day dosing holiday. In some embodiments, dosages are administered daily for 15-20 days followed by a 15-20 day dosing holiday. In some embodiments, dosages are administered daily for 15-20 days followed by a 10-15 day dosing holiday. In some embodiments, dosages are administered daily for 15-20 days followed by a 5-10 day dosing holiday. In some embodiments, dosages are administered daily for 15-20 days followed by a 1-5 day dosing holiday.

In any of the forgoing embodiments, the daily dosing may be administered in one dose administered once or day, or in two or more divided, doses administered multiple times per day. For example, the compounds described herein may be administered once per day, twice per day, three times per day, or four times per day.

Some embodiments of the methods and compositions of the present disclosure are illustrated by the following example.

### Example 1

A fluorescently labeled irreversible activity based probe (ABP) was used to measure off rates of CAPN2 inhibitors as follows. The ABP molecule contains a fluoro-methyl ketone warhead that irreversibly reacts with the active site cysteine nucleophile of CAPN enzymes. ABP is also labeled with the Alexa 647 fluorophore which allows for sensitive quantitation of the CAPN2.ABP adduct.

The assay is typically performed by pre-incubating 2 uM CAPN2 with 1004 test inhibitor for 30 minutes in assay buffer (50 mM Tris-HCl, 100 mM NaCl, 2 mM CaCl₂, 1 mM DTT, 0.02% Brij-35, pH7.4). This incubation mixture (containing the CAPN2.inhibitor complex) is diluted ten-fold into the same assay buffer containing 10 uM ABP. This is time zero at which an aliquot is immediately withdrawn and quenched by adding denaturing SDS-PAGE loading buffer and heating at 95° C. for 5 minutes. Additional aliquots are typically withdrawn and quenched at time points of 0.5, 1, 2, 4, 7, 24 hours. Quenched samples are stored frozen at −80° C. until SDS-PAGE analysis.

For a given test inhibitor, all time points are run on the same gel with the following two control samples. An assay minimum control is prepared by pre-incubating 2 uM CAPN2 in assay buffer for 1 hour. The incubation mixture is diluted, ten-fold into the same assay buffer containing 10 uM ABP, reacted for 30 minutes and quenched. After the initial 1 hour incubation, the CAPN2 enzyme is autolytically degraded and should generate little to no signal. An assay maximum control is prepared by adding 10 uM ABP to 0.2 uM CAPN2 in assay buffer and incubating for 30 minutes and quenching.

After SDS-PAGE, the gel is scanned using a LAS4000 ImageQuant platform to quantify the Alexa 647 intensity at ˜75 kDa (molecular weight of full length activated CAPN2). The minimum and maximum controls on each gel are used to calculate percentage of test compound bound at each time point. This data is fit to a first order decay and the dissociation rate constant (off-rate) obtained. Using the equation t_{1/2}=0.693/k, the off rate (k) is converted to and reported as CAPN2.inhibitor complex half-life (t_{1/2}).

Representative half-lives of target complexes formed by the compounds of the present disclosure are given in Table 1:

TABLE 1
Half Lives of Compound-Target Complexes

| | | Half-life of |
|---|---|---|
| | | Compound- |
| | | CAPN2 |
| | Compound | Complex |
| Compound Structure | No. | (min) |
| 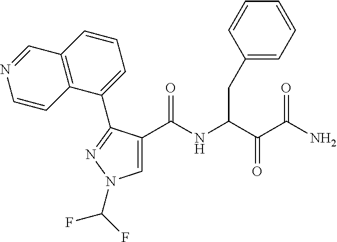 [SMILES: NC(=O)C(=O)C(Cc1ccccc1)NC(=O)c1cn(C(F)F)nc1-c1cccc2cnccc12] | 1 | 168 |
| 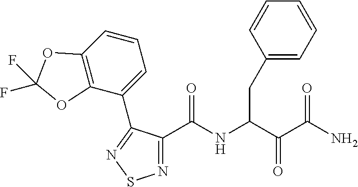 [SMILES: NC(=O)C(=O)C(Cc1ccccc1)NC(=O)c1nsnc1-c1cccc2c1OC(F)(F)O2] | 2 | 578 |
| 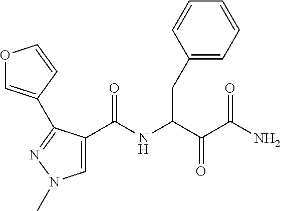 [SMILES: Cn1cc(C(=O)NC(Cc2ccccc2)C(=O)C(N)=O)c(-c2ccoc2)n1] | 3 | fast |
| 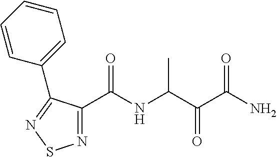 [SMILES: CC(NC(=O)c1nsnc1-c1ccccc1)C(=O)C(N)=O] | 4 | fast |
| 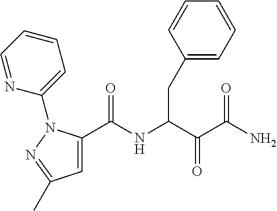 [SMILES: Cc1cc(C(=O)NC(Cc2ccccc2)C(=O)C(N)=O)n(-c2ccccn2)n1] | 5 | 28 |
| 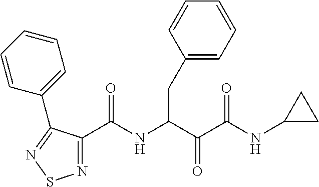 [SMILES: O=C(NC1CC1)C(=O)C(Cc1ccccc1)NC(=O)c1nsnc1-c1ccccc1] | 6 | 23 |
| 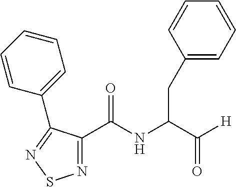 [SMILES: O=CC(Cc1ccccc1)NC(=O)c1nsnc1-c1ccccc1] | 7 | 63 |
| 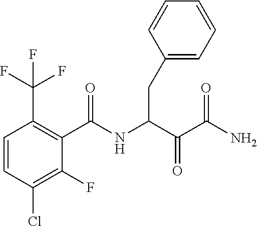 [SMILES: NC(=O)C(=O)C(Cc1ccccc1)NC(=O)c1c(C(F)(F)F)ccc(Cl)c1F] | 8 | 144 |
| 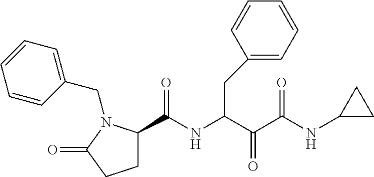 [SMILES: O=C(NC1CC1)C(=O)C(Cc1ccccc1)NC(=O)[C@H]1CCC(=O)N1Cc1ccccc1] | 9 | 5 |
| 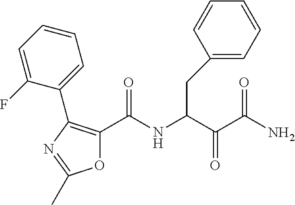 [SMILES: Cc1nc(-c2ccccc2F)c(C(=O)NC(Cc2ccccc2)C(=O)C(N)=O)o1] | 10 | 162 |
| 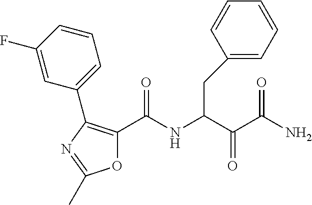 [SMILES: Cc1nc(-c2cccc(F)c2)c(C(=O)NC(Cc2ccccc2)C(=O)C(N)=O)o1] | 11 | 212 |
| 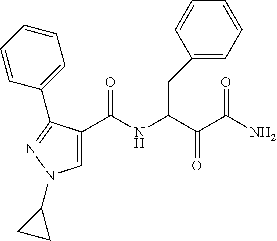 [SMILES: NC(=O)C(=O)C(Cc1ccccc1)NC(=O)c1cn(C2CC2)nc1-c1ccccc1] | 12 | 20 |
| 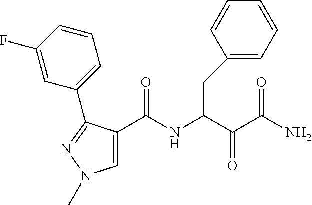 [SMILES: Cn1cc(C(=O)NC(Cc2ccccc2)C(=O)C(N)=O)c(-c2cccc(F)c2)n1] | 13 | 38 |
| 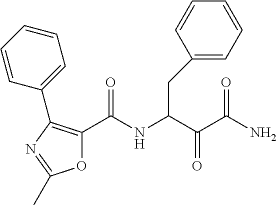 [SMILES: Cc1nc(-c2ccccc2)c(C(=O)NC(Cc2ccccc2)C(=O)C(N)=O)o1] | 14 | 324 |
| 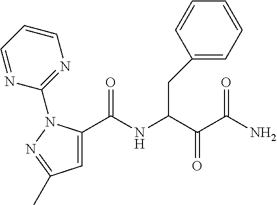 [SMILES: Cc1cc(C(=O)NC(Cc2ccccc2)C(=O)C(N)=O)n(-c2ncccn2)n1] | 15 | fast |
| 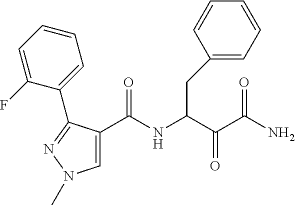 [SMILES: Cn1cc(C(=O)NC(Cc2ccccc2)C(=O)C(N)=O)c(-c2ccccc2F)n1] | 16 | 52 |
| 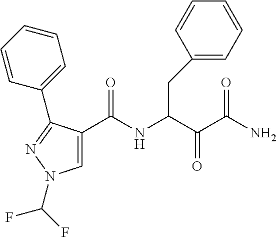 [SMILES: NC(=O)C(=O)C(Cc1ccccc1)NC(=O)c1cn(C(F)F)nc1-c1ccccc1] | 17 | 126 |
| 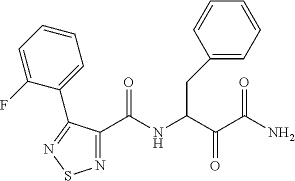 [SMILES: NC(=O)C(=O)C(Cc1ccccc1)NC(=O)c1nsnc1-c1ccccc1F] | 18 | 330 |
| 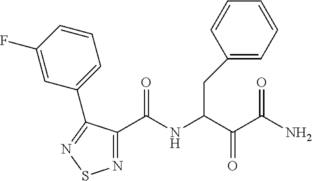 [SMILES: NC(=O)C(=O)C(Cc1ccccc1)NC(=O)c1nsnc1-c1cccc(F)c1] | 19 | 168 |
| 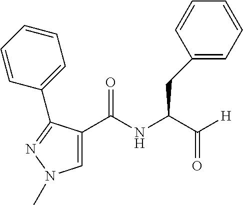 [SMILES: Cn1cc(C(=O)N[C@H](C=O)Cc2ccccc2)c(-c2ccccc2)n1] | 20 | fast |
| 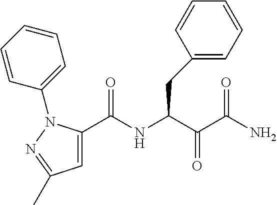 [SMILES: Cc1cc(C(=O)N[C@@H](Cc2ccccc2)C(=O)C(N)=O)n(-c2ccccc2)n1] | 21 | 162 |
| 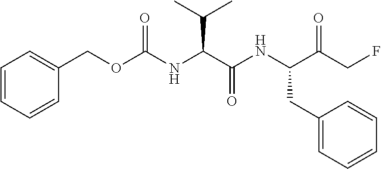 [SMILES: CC(C)[C@H](NC(=O)OCc1ccccc1)C(=O)N[C@@H](Cc1ccccc1)C(=O)CF] | 22 | irreversible |
| 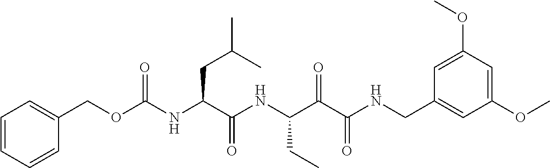 [SMILES: CC[C@H](NC(=O)[C@H](CC(C)C)NC(=O)OCc1ccccc1)C(=O)C(=O)NCc1cc(OC)cc(OC)c1] | 23 | 60 |
| 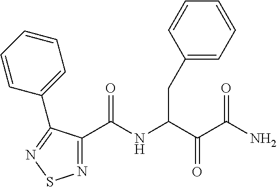 [SMILES: NC(=O)C(=O)C(Cc1ccccc1)NC(=O)c1nsnc1-c1ccccc1] | 24 | 396 |
| 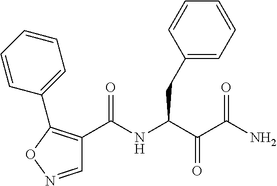 [SMILES: NC(=O)C(=O)[C@H](Cc1ccccc1)NC(=O)c1cnoc1-c1ccccc1] | 25 | 96 |
| 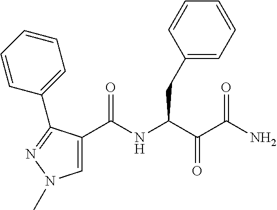 [SMILES: Cn1cc(C(=O)N[C@@H](Cc2ccccc2)C(=O)C(N)=O)c(-c2ccccc2)n1] | 26 | 29 |
| 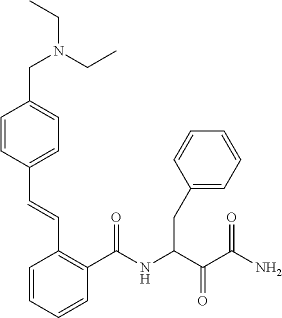 [SMILES: CCN(CC)Cc1ccc(/C=C/c2ccccc2C(=O)NC(Cc2ccccc2)C(=O)C(N)=O)cc1] | 27 | 54 |
| 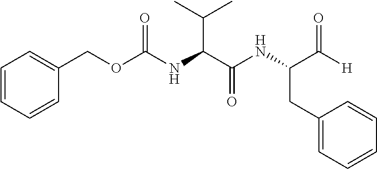 [SMILES: CC(C)[C@H](NC(=O)OCc1ccccc1)C(=O)N[C@H](C=O)Cc1ccccc1] | 28 | 180 |
| 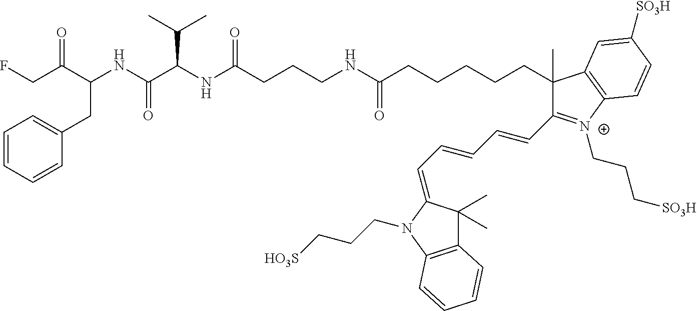 [SMILES: CC(C)[C@@H](NC(=O)CCCNC(=O)CCCCCC1(C)C(/C=C/C=C/C=C2/N(CCCS(=O)(=O)O)c3ccccc3C2(C)C)=[N+](CCCS(=O)(=O)O)c2ccc(S(=O)(=O)O)cc21)C(=O)NC(Cc1ccccc1)C(=O)CF] | 29 | 420 |
| 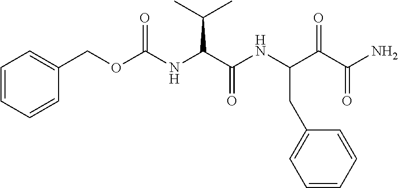 | 30 | 1066 |
| 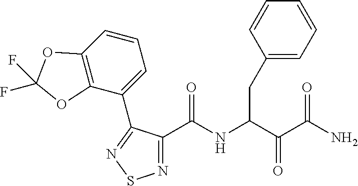 [SMILES: NC(=O)C(=O)C(Cc1ccccc1)NC(=O)c1nsnc1-c1cccc2c1OC(F)(F)O2] | 31 | 292 |
| 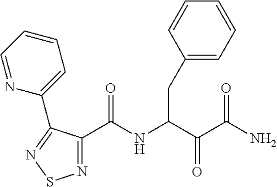 [SMILES: NC(=O)C(=O)C(Cc1ccccc1)NC(=O)c1nsnc1-c1ccccn1] | 32 | 139 |
| 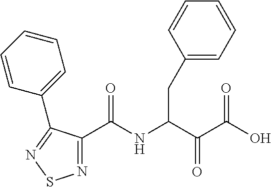 [SMILES: O=C(O)C(=O)C(Cc1ccccc1)NC(=O)c1nsnc1-c1ccccc1] | 33 | 112 |
| 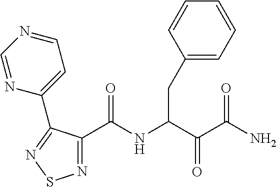 [SMILES: NC(=O)C(=O)C(Cc1ccccc1)NC(=O)c1nsnc1-c1ccncn1] | 34 | 76 |
| 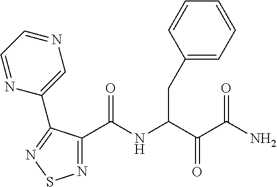 [SMILES: NC(=O)C(=O)C(Cc1ccccc1)NC(=O)c1nsnc1-c1cnccn1] | 35 | 62 |
| 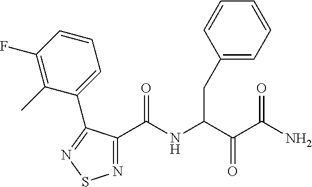 [SMILES: Cc1c(F)cccc1-c1nsnc1C(=O)NC(Cc1ccccc1)C(=O)C(N)=O] | 36 | 45 |
| 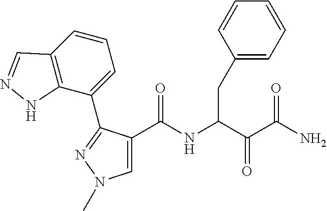 [SMILES: Cn1cc(C(=O)NC(Cc2ccccc2)C(=O)C(N)=O)c(-c2cccc3cn[nH]c23)n1] | 37 | 39 |
| 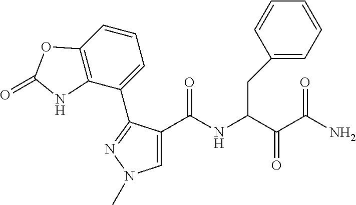 [SMILES: Cn1cc(C(=O)NC(Cc2ccccc2)C(=O)C(N)=O)c(-c2cccc3oc(=O)[nH]c23)n1] | 38 | 39 |
| 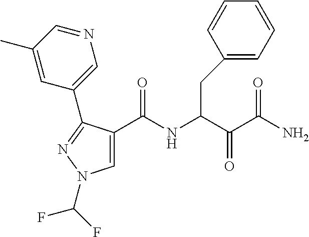 [SMILES: Cc1cncc(-c2nn(C(F)F)cc2C(=O)NC(Cc2ccccc2)C(=O)C(N)=O)c1] | 39 | 32 |
| 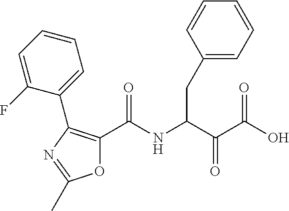 [SMILES: Cc1nc(-c2ccccc2F)c(C(=O)NC(Cc2ccccc2)C(=O)C(=O)O)o1] | 40 | 28 |
| 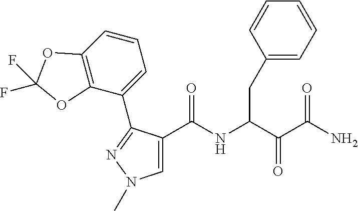 [SMILES: Cn1cc(C(=O)NC(Cc2ccccc2)C(=O)C(N)=O)c(-c2cccc3c2OC(F)(F)O3)n1] | 41 | 27 |

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” Should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges Which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of treating a disease or condition, comprising the steps, in order, of:
administering for a first number of days to a subject in need thereof a first daily amount of one or more compounds having the structure of formula (I) or formula (II): 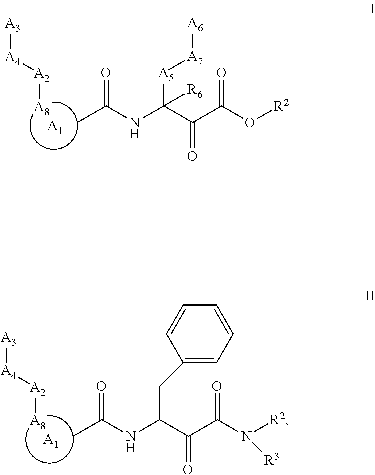
or a pharmaceutically acceptable salt thereof, wherein:
A₁ is selected from the group consisting of optionally substituted 5-10 membered heterocyclyl; optionally substituted 5-, 8-, or 9-membered heteroaryl; and optionally substituted C₃₋₁₀ carbocyclyl, provided that in compounds of formula (II), A₁ is not a 5-10 membered heterocyclyl substituted with oxo;
A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH═CH—, —C≡C—, —OC(O)NH—, —NHC(O)NH—, —NHC(O)O—, —NHC(O)—, —NHC(S)NH—, —NHC(S)O—, —NHC(S)—, and single bond;
A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;
when A₂ and A₄ are single bond, A₃ is directly attached to A₈;
A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;
A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —OC₂₋₆ alkenyl, —OSO₂CF₃, and any natural or non-natural amino acid side chain;
A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
when A₅ and A₇ are single bond, A₆ is directly attached to the carbon to which R⁸ is attached;
A₈ is a ring member of A₁ and is selected from the group consisting of C and N;
R is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;
R² in formula (I) is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;
R² and R³ in formula (II) are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;
R⁶ is independently selected from —H and optionally substituted C₁₋₄ alkyl; and
each n is independently selected to be an integer from 0 to 3; or any combination thereof; or pharmaceutically acceptable salts thereof;
ceasing administration of the compound or administering a second daily amount of the compound for a second number of days, wherein the second daily amount of the compound is less than the first daily amount; and
administering a third daily amount of the compound for a third number of days to the subject.

2. (canceled)

3. A method of treating a disease or condition, comprising the steps, in order, of:
administering for a first number of days to a subject in need thereof a first daily amount of a compound having a structure selected from the group consisting of: 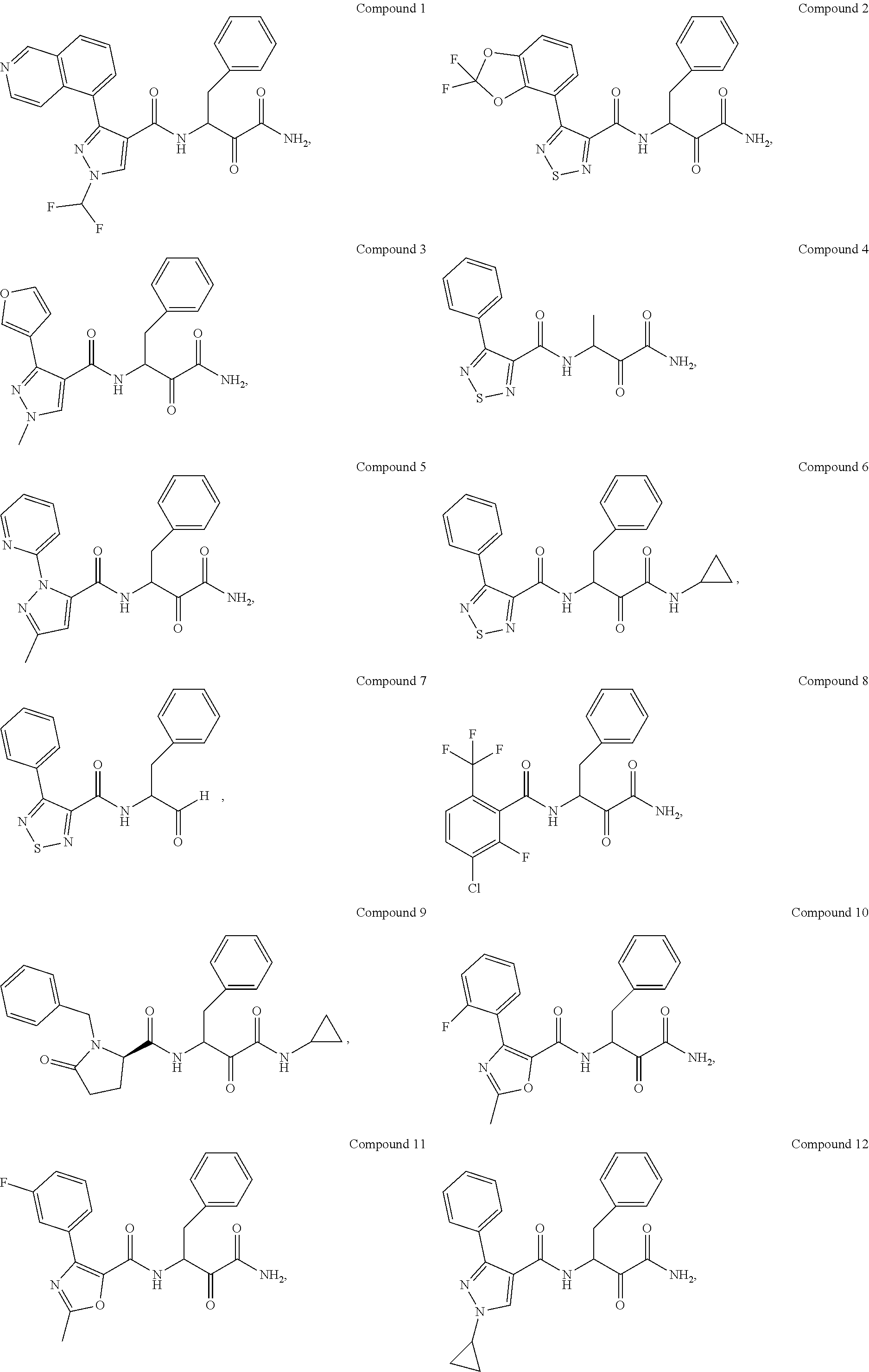 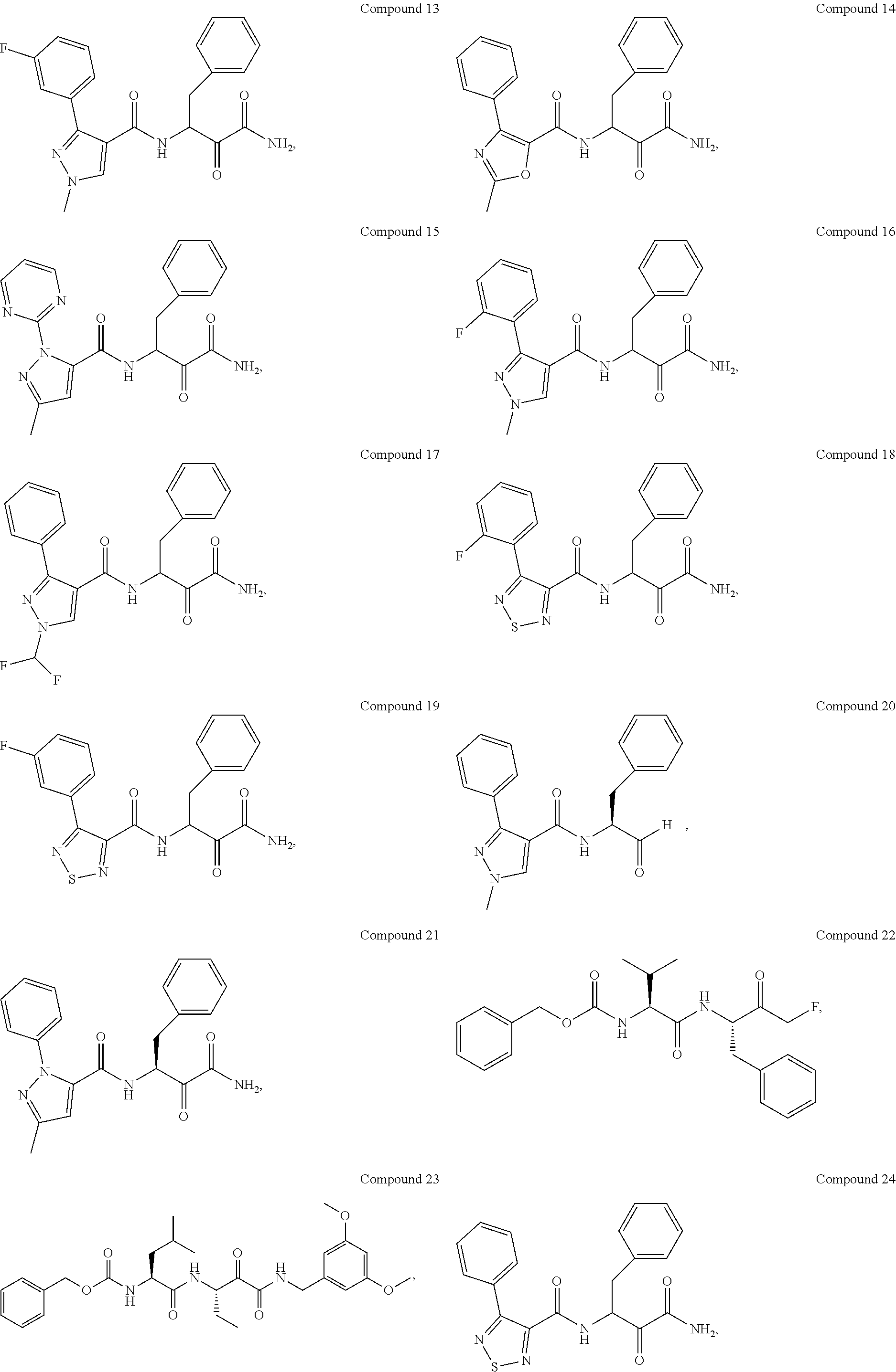 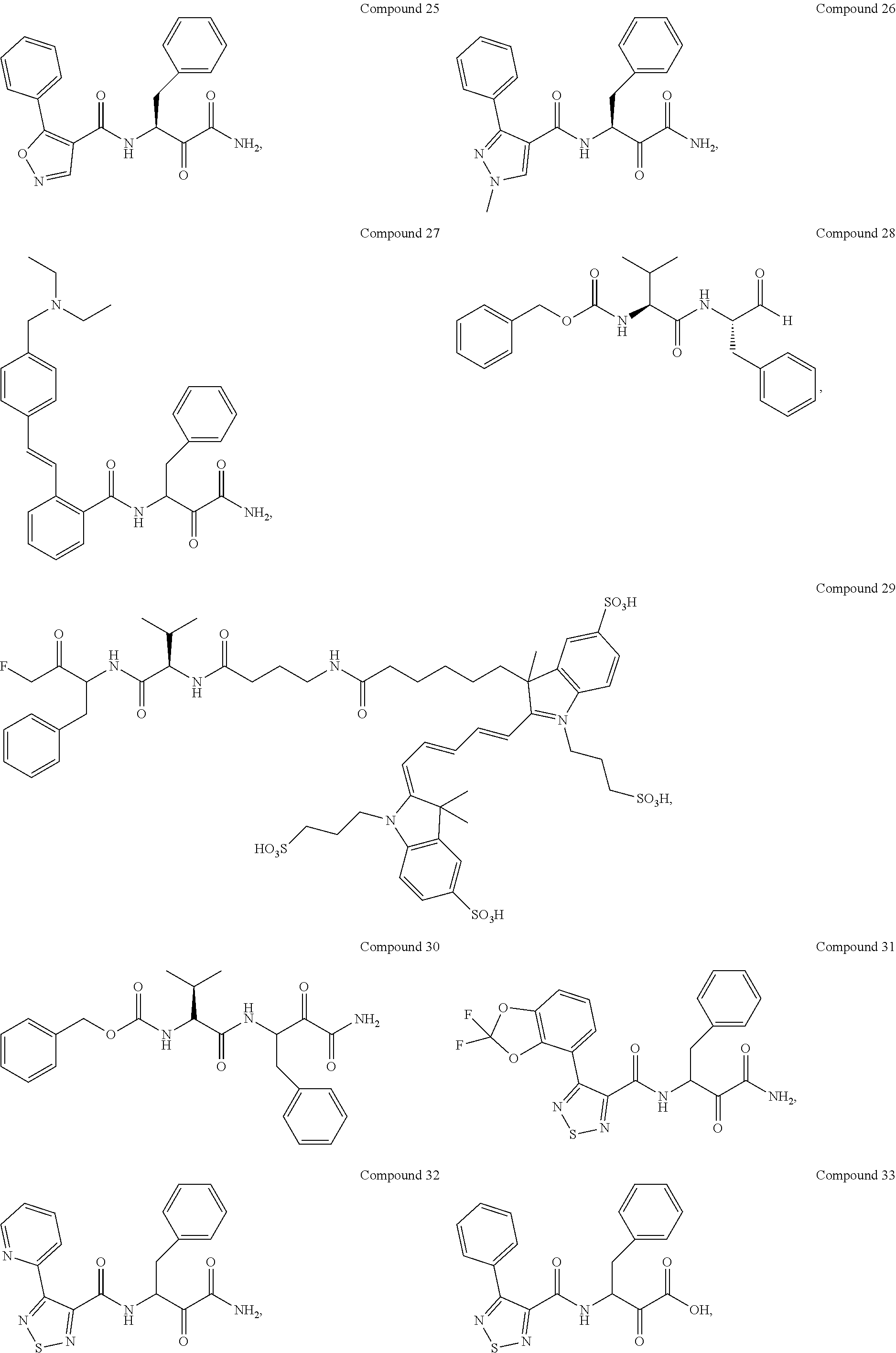 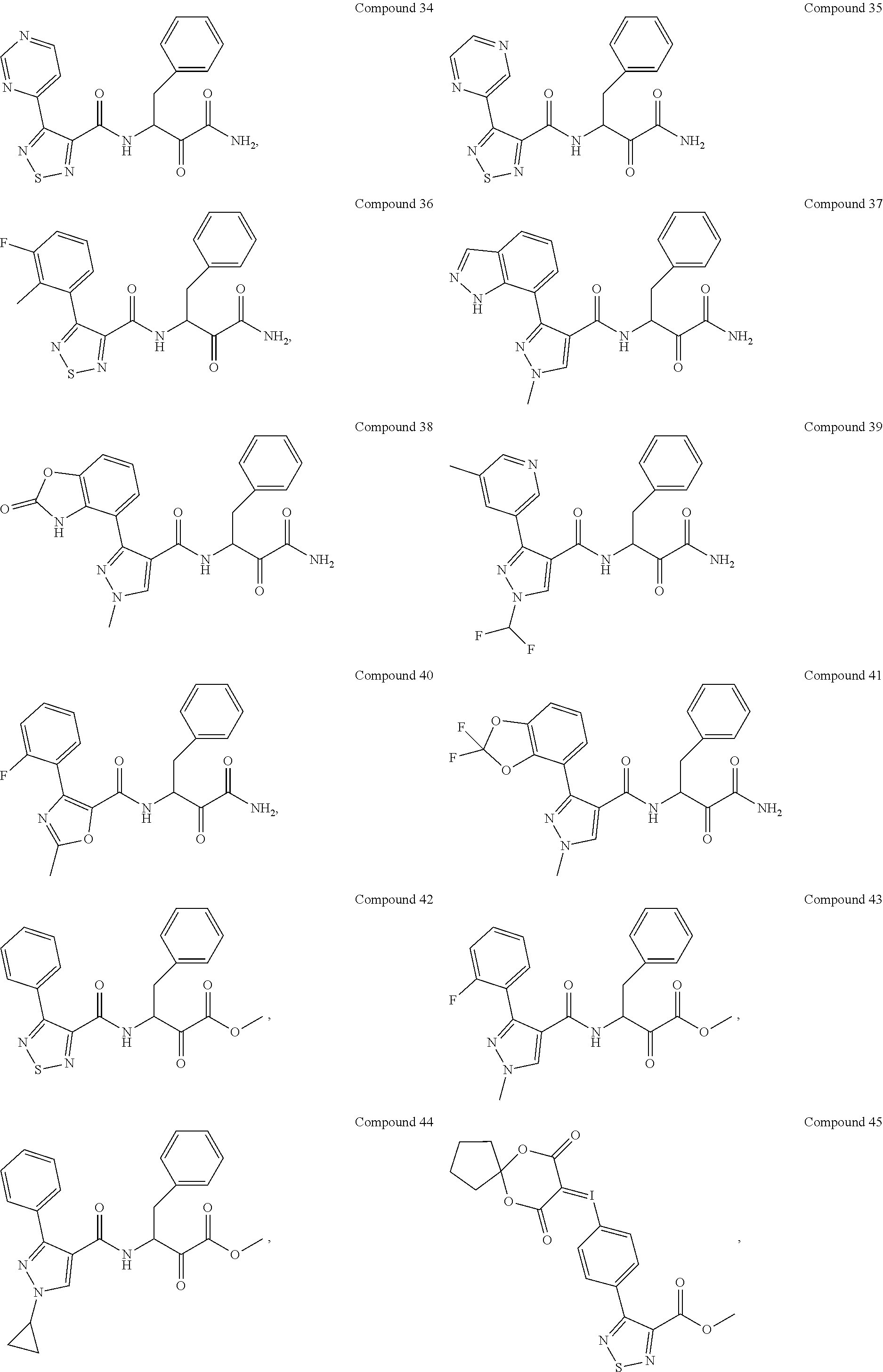 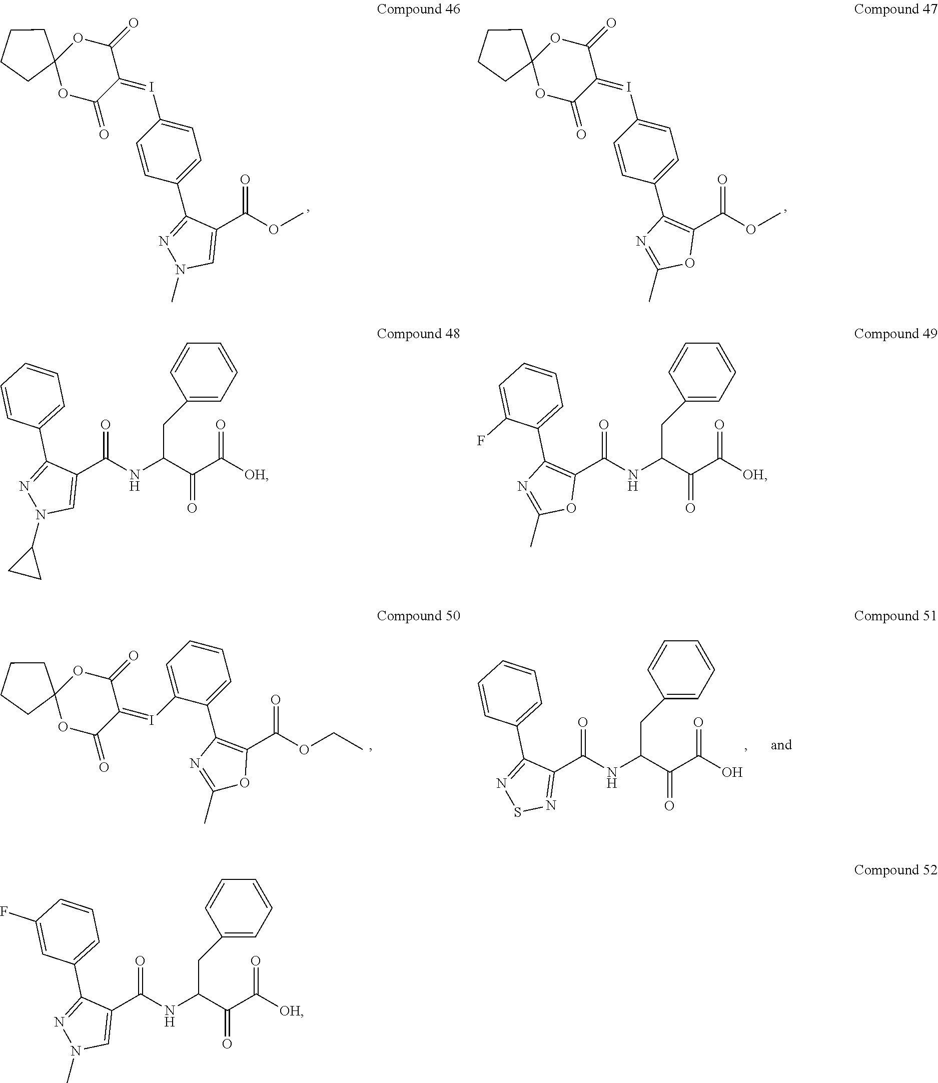
and any combination thereof; or pharmaceutically acceptable salts thereof;
ceasing administration of the compound or administering a second daily amount of the compound for a second number of days, wherein the second daily amount of the compound is less than the first daily amount; and
administering a third daily amount of the compound for a third number of days to the subject.

4. The method of claim 1, wherein the first and third daily amounts are the same or the third daily amount is less than the first daily amount.

5. (canceled)

6. The method of claim 1, wherein the compound is administered once, twice, three times, or four times per week.

7. (canceled)

8. The method of claim 1, wherein the compound is administered every other day, every third day, every fourth day, every fifth day or every sixth day.

9-12. (canceled)

13. The method of claim 1, wherein the second and third daily amounts are the same or the third daily amount is greater than the second daily amount.

14. (canceled)

15. The method of claim 1, wherein the first and third number of days are the same or the first, second, and third number of days are the same.

16. (canceled)

17. The method of claim 1, wherein the third number of days is less than the first number of days.

18. The method of claim 1, wherein the first, second, and third number of days are independently selected from 1 to 5, 1 to 10, 1 to 20, 1 to 30 and 1 to 90.

19-22. (canceled)

23. The method of claim 1, wherein the first and third number of days is 1 and the second number of days is 1 or 2.

24. (canceled)

25. The method of claim 1, wherein the first and third number of days is 3 and the second number of days is 4.

26. The method of claim 1, wherein the first and third number of days is 4 and the second number of days is 3, 4 or 5.

27. (canceled)

28. The method of claim 1, wherein the first and third number of days is 5 and the second number of days is 4.

29. (canceled)

30. The method of claim 1, wherein the first and third number of days is 10 and the second number of days is 10 or the first and third number of days is 30 and the second number of days is 30.

31. (canceled)

32. The method of claim 1, wherein the first and third number of days is 2 and the second number of days is 1.

33. The method of claim 1, wherein the administration during the first and third number of days is once per day.

34. The method of claim 1, comprising ceasing administration of the compound for the second number of days.

35. The method of claim 1, comprising administering the second daily amount of the compound for the second number of days.

36. The method of claim 1, comprising monitoring the subject's levels of any of said compounds and ceasing administration of said compound or administering the second daily amount of said compound when the level of said compound are above a first threshold value and resuming administration of the compound at the first daily amount when the level of said compound is below a second threshold value.

37. The method of claim 36, wherein the first and second threshold values are the same.

38. The method of claim 1, wherein the total weekly dosage of the compound during the first number of days is from 5 to 250 mg, 40 to 150 mg, 50 to 90 mg or 60 to 80 mg.

39-41. (canceled)

42. The method of claim 1, wherein the maximum serum concentration of the compound is 100 ng/mL or less during the third number of days or during the entire treatment period.

43-44. (canceled)

45. The method of claim 1, comprising:
ceasing administration of the compound or administering the second daily amount of the compound for a fourth number of days;
administering the third daily amount of the compound for a fifth number of days; and
repeating said ceasing administration or administering the second daily amount for the fourth number of days, and said administering the third daily amount of the compound for the fifth number of days.

46. The method of claim 1, wherein said disease or condition comprises a fibrotic condition.

47. The method of claim 1, wherein said disease or condition comprises one or more of liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases or disorders or any symptom or sequela thereof, or any combination thereof.

48. A method of treating a disease or condition, comprising the steps, in order, of:
Administering to a subject in need thereof a loading dose for a first period of time one or more compounds having the structure of the formula (I) or the formula (II): 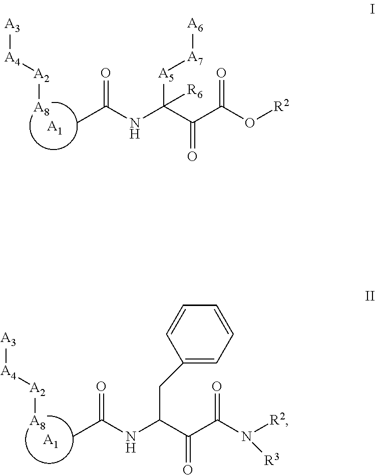
or a pharmaceutically acceptable salt thereof, wherein:
A₁ is selected from the group consisting of optionally substituted 5-10 membered heterocyclyl; optionally substituted 5-, 8-, or 9-membered heteroaryl; and optionally substituted C₃₋₁₀ carbocyclyl, provided that in compounds of formula (II), A₁ is not a 5-10 membered heterocyclyl substituted with oxo;
A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH═CH—, —C≡C—, —OC(O)NH—, —NHC(O)NH—, —NHC(O)O—, —NHC(O)—, —NHC(S)NH—, —NHC(S)O—, —NHC(S)—, and single bond;
A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;
when A₂ and A₄ are single bond, A₃ is directly attached to A₈;
A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;
A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —OC₂₋₆ alkenyl, —OSO₂CF₃, and any natural or non-natural amino acid side chain;
A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
when A₅ and A₇ are single bond, A₆ is directly attached to the carbon to which R⁸ is attached;
A₈ is a ring member of A₁ and is selected from the group consisting of C and N;
R is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;
R² in formula (I) is selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;
R² and R³ in formula (II) are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and
R⁶ is independently selected from —H and optionally substituted C₁₋₄ alkyl; and
each n is independently selected to be an integer from 0 to 3; or any combination thereof; or pharmaceutically acceptable salts thereof; and
administering a maintenance dose of the compound for a second period of time to the subject.

49. (canceled)

50. A method of treating a disease or condition, comprising the steps, in order, of:
administering to a subject in need thereof a loading dose for a first period of time a compound having a structure selected from the group consisting of: 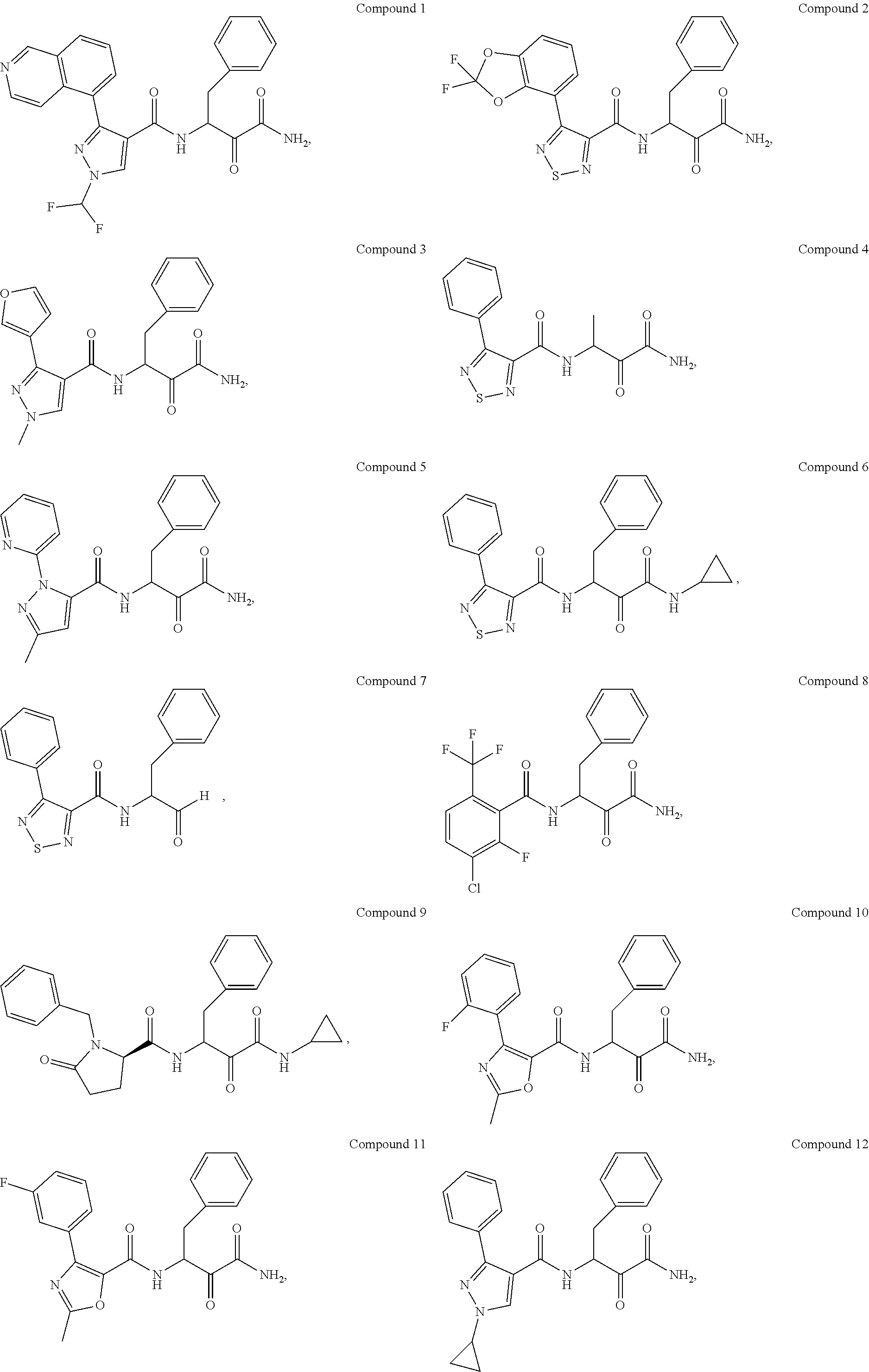 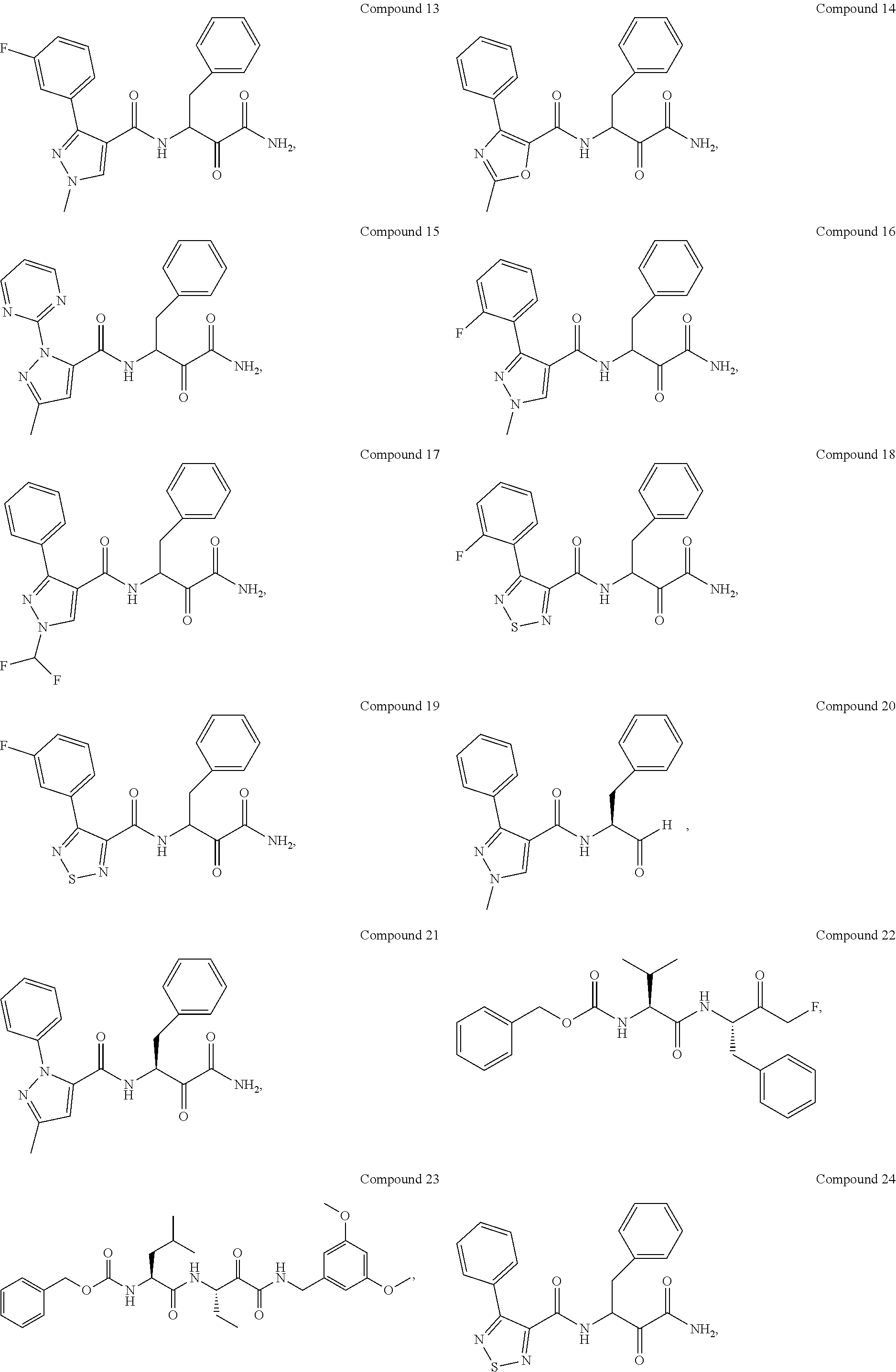 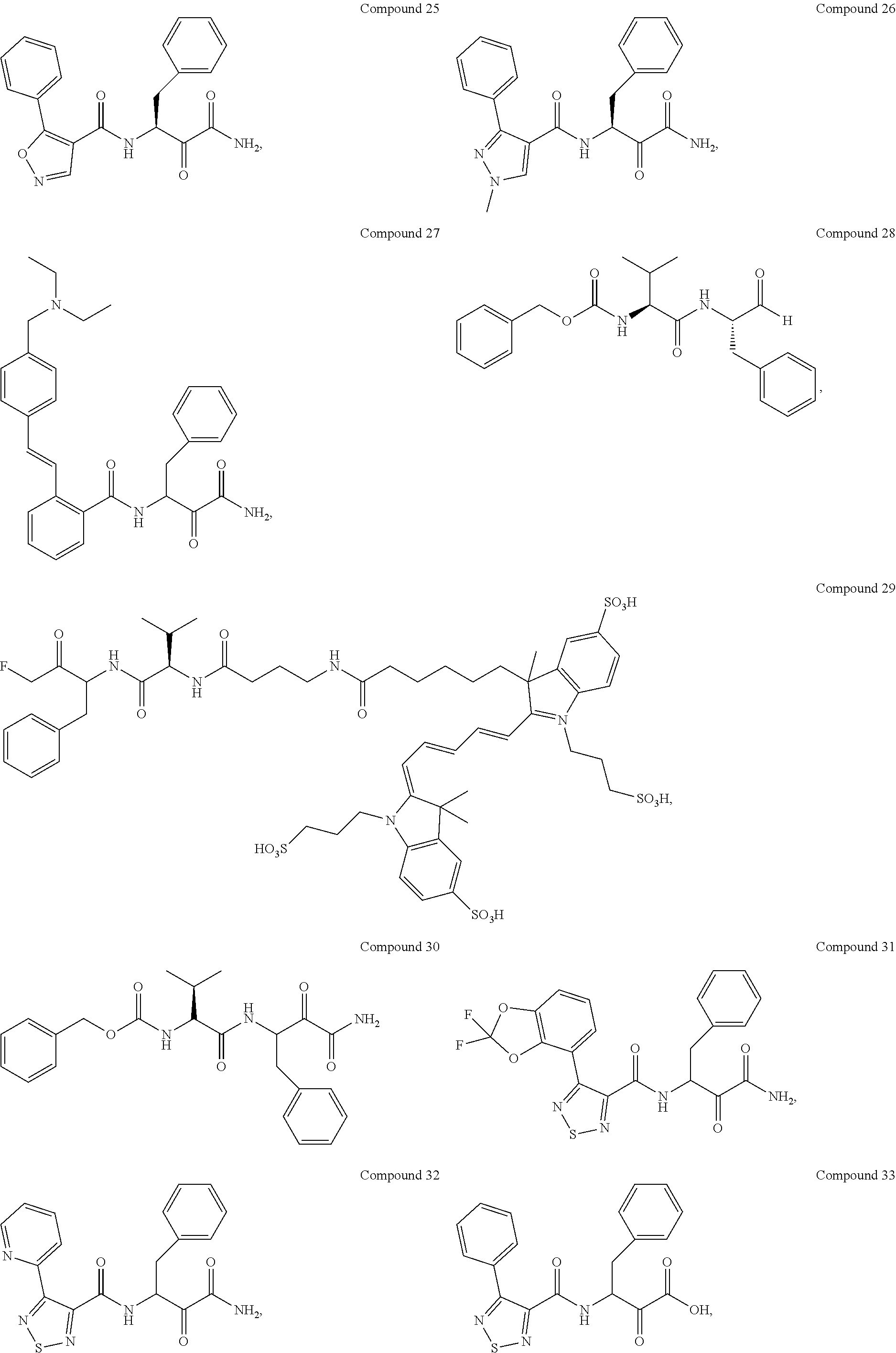 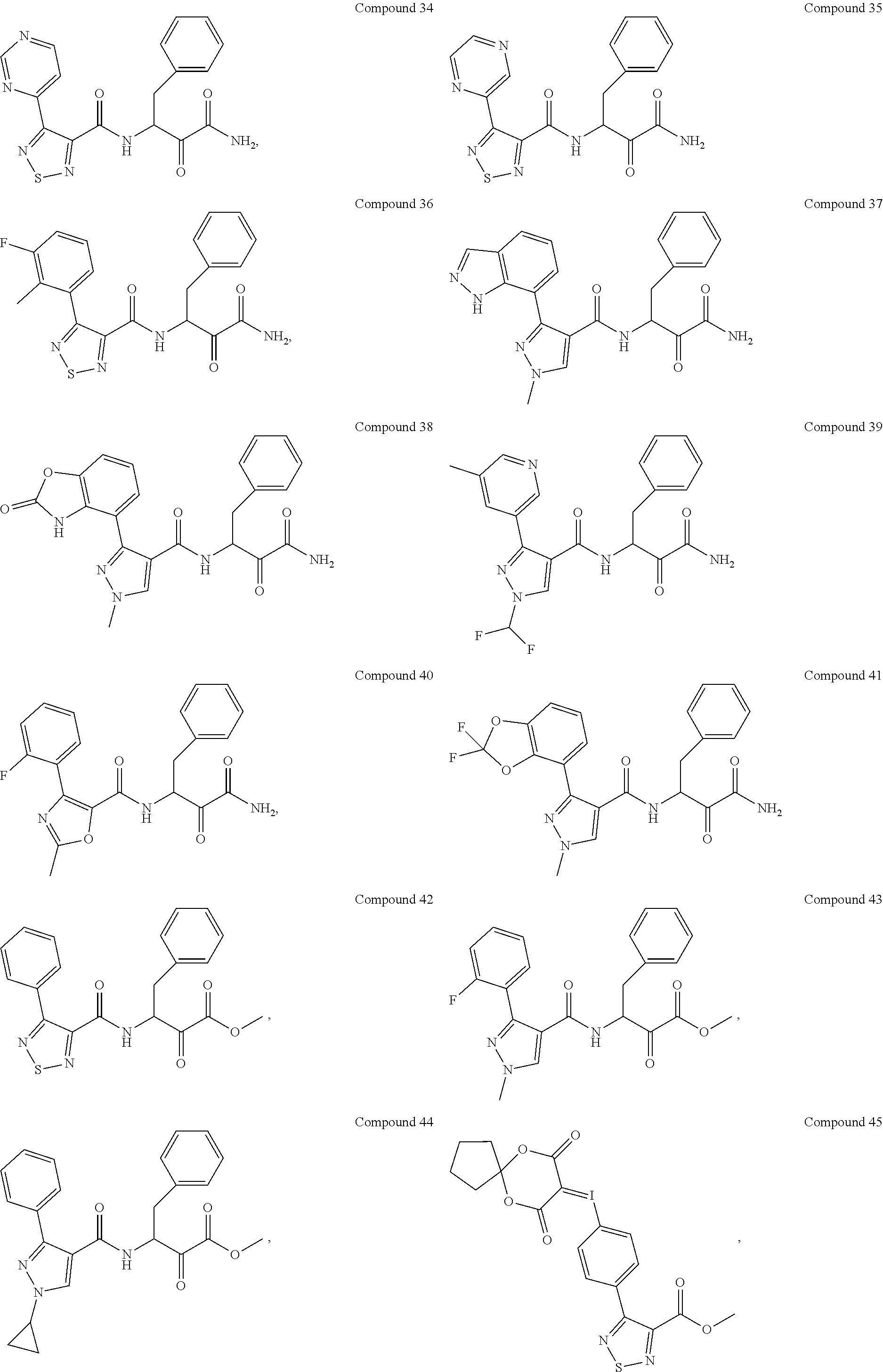 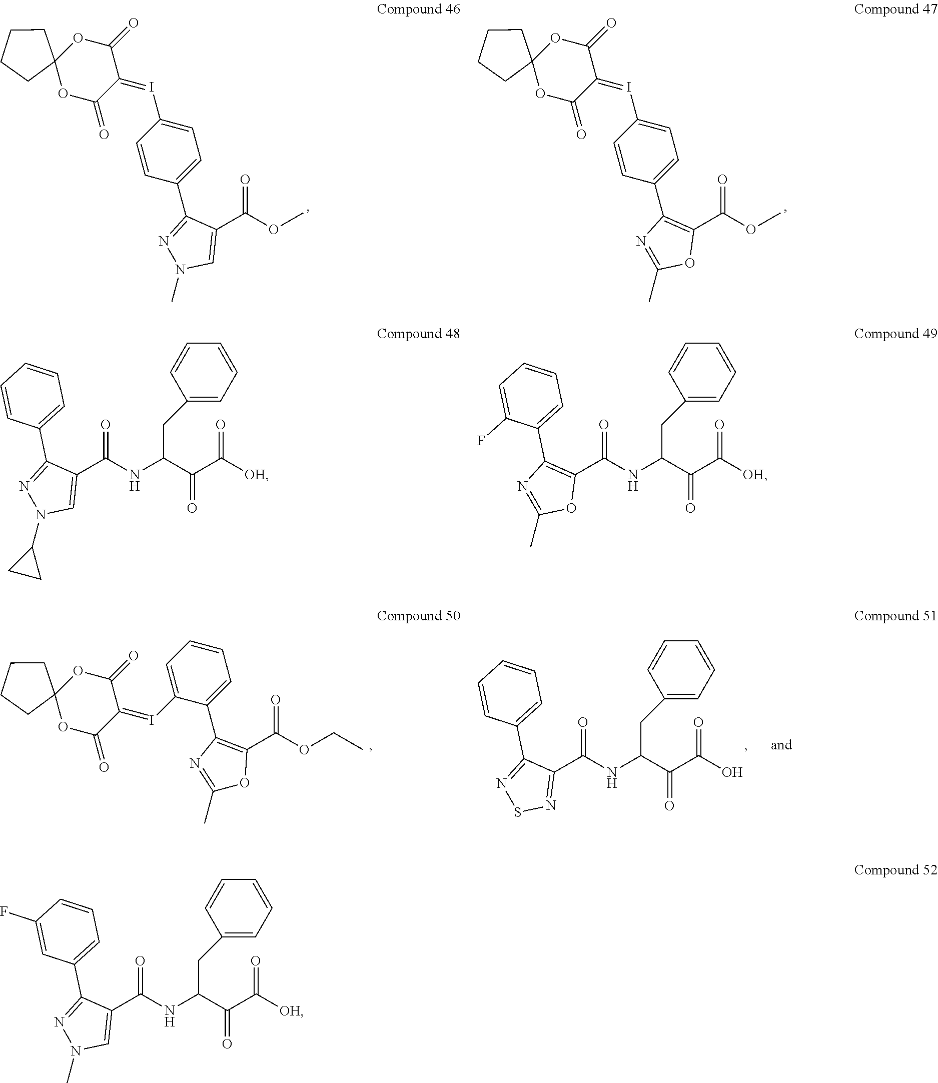
and any combination thereof; or pharmaceutically acceptable salts thereof; and
administering a maintenance dose of the compound for a second period of time to the subject.

51. The method of claim 48, wherein the first period of time is 1-7 days or 8-14 days.

52. (canceled)

53. The method of claim 48, wherein the second period of time is 14 days or greater.

54. The method of claim 48, wherein the loading dose is administered once per day or twice per day.

55. (canceled)

56. The method of claim 48, wherein the maintenance dose is administered once per day, twice per day, every other day or once per week.

57-59. (canceled)

60. The method of claim 48, wherein said disease or condition comprises a fibrotic condition.

61. The method of claim 48, wherein said disease or condition comprises one or more of liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases or disorders or any symptom or sequela thereof, or any combination thereof.

62-64. (canceled)
